(19) United States
(12) Patent Application Publication
(10) Pub. No.: US 2005/0164335 A1
(43) Pub. Date: Jul. 28, 2005

(54) Methods and organisms for production of b6 vitamers

(76) Inventors: R Rogers Yocum, Lexington, MA (US); Mark K. Williams, Revere, MA (US); Janice G. Pero, Lexington, MA (US)

Correspondence Address: LAHIVE & COCKFIELD, LLP., 28 STATE STREET, BOSTON, MA, 02109, US

(21) Appl. No.: 10/508,768

(22) PCT Filed: Mar. 21, 2003
(86) PCT No.: PCT/US03/08880

Related U.S. Application Data
(60) Provisional application No. 60/367,863, filed on Mar. 25, 2002.
(60) Provisional application No. 60/368,618, filed on Mar. 29, 2002.

(30) Foreign Application Priority Data
Mar. 22, 2002 (US) 60367089

(51) Int. Cl. C12P 25/00; C12P 17/12; C12N 9/10; C12N 1/12; C12N 1/18; C12N 1/21

(52) U.S. Cl. 435/66; 435/122; 435/193; 435/252.3; 435/254.1; 435/257.1

(57) ABSTRACT

The present invention features methods of producing B6 vitamers that involve culturing an organism overexpressing an enzyme that catalyzes a step in the biosynthesis of a B6 vitamer under conditions such that a B6 vitamer is produced. The present invention further features methods of producing B6 vitamers that involve culturing recombinant microorganisms having increased activity of at least one B6 vitamer biosynthetic enzyme, e.g., YaaD or YaaE, or a homologue thereof, or Epd, PdxA, PdxJ, PdxF, PdxB, PdxH, and/or Dxs, or a homologue thereof.

### RELATED APPLICATIONS

This application claims priority to U.S. Provisional Application Ser. No. ______ entitled “METHODS AND ORGANISMS FOR PRODUCTION OF B6 VITAMERS” (Atty Ref. No. BGI-152-4), filed on Mar. 3, 2003, U.S. Provisional Application Ser. No. 60/367,089 filed on Mar. 22, 2002, U.S. Provisional Application Ser. No. 60/367,863 filed on Mar. 25, 2002, and U.S. Provisional Application Ser. No. 60/368,618 filed on Mar. 29, 2002. The entire content of the above-referenced applications is incorporated herein by this reference.

### BACKGROUND OF THE INVENTION

Vitamin B6, also known as pyridoxine or pyridoxol (PN), or one of a number of closely related compounds, is an essential dietary nutrient for most, if not all, animals, while many microorganisms (bacteria, fungi, algae, etc.) and plants are capable of synthesizing their own vitamin B6 or compound(s) related to vitamin B6. When an animal ingests PN or a related compound that has vitamin B6 activity, the compound is converted ultimately into pyridoxal phosphate (PLP) and/or pryidoxamine phosphate (PMP), which are the active forms of vitamin B6 in all living organisms. PLP acts as a cofactor for many important or essential enzymes in all living organisms, including transaminases, racemases, and decarboxylases. PLP and PMP are easily interconverted by ubiquitous transaminases.

Vitamin B6 is of commercial importance in vitamin pills, pharmaceutical applications, and as an animal feed additive that enhances growth or desirable growth characteristics in farm and domestic animals. The currently used commercial process for producing vitamin B6 is a synthetic chemical process. However, a fermentation process using a microorganism (see U.S. patent application Ser. No. 09/667,569, filed Sep. 21, 2000, hereby incorporated in its entirety by reference) or a biosynthetic process using a plant species can be more cost effective in the long run, and may be environmentally more attractive.

The biosynthetic pathway for PLP in E. coli has been elucidated (reviewed in Mittengruber, G., (2001) J. Mol. Microbiol. Biotechnol. 3(1): 1-20; Cane, D. E., et al. (2000) J. Am. Chem. Soc. 122: 4213-4214; Man, T-K, et al., (1996) J. Bacteriol. 178: 2445-2449). Enzymes encoded by the genes epd, pdxB, pdxF, and pdxA lead to synthesis of the precursor 1-hydroxy-3-amino acetone phosphate from erythrose-4-phosphate and glutamate. The enzyme encoded by dxs leads to the precursor 5′-deoxyxylulose phosphate from glycolytic intermediates. The enzyme encoded by pdxJ then catalyzes the chemical coupling of the two precursors to give pyridoxol phosphate (also called pyridoxine phosphate or PNP). PNP is then oxidized to the active form, PLP, by the enzyme encoded by pdxH. This biosynthetic pathway to PLP in E. coli, as well as closely related pathways, are referred to herein as the Type A Pathway. Partially characterized mutants of E. coli have been described that produce about three- to seven-fold more vitamin B₆-related compounds than the parent strain (Dempsey and Arcement (1971) J. Bacteriol. 107(2): 580-582). Partially characterized mutants of B. subtilis have been reported that produce 1-5 mg/l vitamin B₆, but it was not stated what level the parent strain produced (Pflug, W., and Lingens, F., (1978) Hoppe-Seyler's Z. Physiol. Chem. 359: 559-570). Notably, these organisms were not recombinantly produced.

A second biosynthetic pathway for vitamin B6, referred to herein as the Type B pathway, may exist in some organisms other than E. coli (Mittengruber, G., (2001) J. Mol. Microbiol. Biotechnol. 3(1): 1-20). In particular, some fingi (for example from the genera Cercospora, Neurospora, Aspergillus and Saccharomyces), some bacteria (for example B. subtilis and Staphylococcus aureus), and all plants for which data exists do not contain any genes that are highly homologous to E. coli pdxA and pdxJ. Instead, these organisms contain genes that are homologous to Cercospora genes named SOR (or SNZ) and SNO. In Saccharomyces, these homologs are called PDX1 and PDX2, respectively, and in B. subtilis, these homologs are named yaaD and yaaE, respectively. Protein or DNA sequence homology alone is not sufficient to establish biological function. For example, B. subtilis contains a gene, yhaF, that encodes a protein that is significantly homologous to E. coli pdxF. However, when yhaF is mutated, the resulting mutant B. subtilis strain is a serine auxotroph, but not a PL auxotroph (see Example 3, below). Thus, the identification of a gene or genes involved in PLP biosynthesis in any given organism can not be done using sequence homology alone. However, the identification of sequence homology between genes, in combination with the presence of one or more common biological activities, may be used to identify homologs of genes involved in PLP biosynthesis.

Results from ¹³C and ¹⁵N labeling studies suggest that the precursors that provide the carbon and nitrogen atoms in PL and related compounds are different in E. coli and Saccharomyces cerevisiae (Gupta, R., et al. (2001) J. Am Chem. Soc. 123: 11353-11359; Tayuza, K., et al. (1995) Biochim. Biophys. Acta 1244: 113-116.) However, the identity of the precursors for PL and related compounds in S. cerevisiae is not yet known. Since most microorganisms for which the entire genome sequence is known (for example E. coli, S. cerevisiae and B. subtilis) have either pdxA and pdxJ homologs or SOR and SNO homologs, but not both, it appears that most organisms that are capable of synthesizing PLP have either the well characterized Type A Pathway (for example E. coli, Salmonella typhimurium, and many other genera), or a distinctly different and incompletely characterized pathway, e.g., the Type B Pathway. Specifically, members of the genera Cercospora, Neurospora, Aspergillus, Saccharomyces, Bacillus, Arabidopsis, and many other genera, appear to have a Type B pathway, and are lacking some of the genes involved in the Type A Pathway. The intermediate compounds in the Type B Pathway have not yet been elucidated, although the final product must be PLP (as for the Type A Pathway) or PMP, since these are the active forms of vitamin B6 in all known organisms.

### SUMMARY OF THE INVENTION

The present invention is based, at least in part, on the discovery of key enzyme-encoding genes of the B6 vitamer biosynthetic pathways in, e.g., Bacillus subtilis. In particular, the invention is based, at least in part, on the discovery that the yaaD and yaaE genes of B. subtilis, or homologues thereof, are required for B6 vitamer synthesis. The invention is further based on the discovery that the biosynthesis of vitamers by an organism can be increased by increasing the level of one or more enzymes involved in B6 vitamer synthesis.

Deletion of a portion of the yaaD and yaaE genes (which are adjacent in an operon, e.g., the yaaDE operon) leads to PL auxotrophy. Overexpression of the yaaDE operon or the deregulation of the expression of the yaaD and yaaE genes leads to significantly increased production of B6 vitamers in, e.g., B. subtilis strains. The B. subtilis yaaDE operon is required for pyridoxal phosphate (PLP) biosynthesis, an active form of vitamin B6 in all living organisms. The present invention describes that the expression of the B. subtilis yaaDE operon is a rate limiting step for production of compounds related to vitamin B6 in a wild type strain.

Accordingly, the present invention features methods of producing B6 vitamers, including, but not limited to, pyridoxine (or pyridoxol (PN)), pyridoxal (PL), pyridoxamine (PM), or the 5′ phosphorylated derivatives of any of the three aforementioned compounds (PNP, PLP, and PMP), using organisms in which the B6 vitamer pathway has been manipulated such that B6 vitamers are produced. Such methods include culturing an organism, e.g., a microorganism that overexpresses at least one B6 vitamer biosynthetic enzyme under conditions such that the B6 vitamer is produced.

Based on the discovery of the activity of the polypeptides encoded by the yaaD and yaaE genes, or homologues thereof, in B6 vitamer synthesis, the invention is also based, at least in part, on the discovery that modulation of YaaD and/or YaaE activity (e.g., YaaD and YaaE activity or YaaD activity) results in the modulation of BE vitamer production. Accordingly, in one aspect, the invention includes a method for producing a B6 vitamer comprising culturing an organism with an increased YaaD and/or YaaE activity (e.g., YaaD and YaaE activity or YaaD activity) as compared to the parent organism. In one embodiment, increased YaaD and/or YaaE activity (e.g., YaaD and YaaE activity or YaaD activity) is due to increased expression of a nucleic acid molecule encoding a YaaD polypeptide and/or YaaE polypeptide (e.g., a YaaD polypeptide and a YaaE polypeptide or a YaaD polypeptide) as compared to an unmodified parent organism. In another embodiment, increased yaaD and/or yaaE nucleic acid molecule expression (e.g., yaaD and yaaE nucleic acid expression or yaaD nucleic acid expression), is due to the introduction of nucleic acid molecules encoding a YaaD polypeptide and/or a YaaE polypeptide into the organism.

In another aspect, the invention provides methods for producing a B6 vitamer comprising culturing an organism with an increased Epd, PdxA, PdxJ, PdxF, PdxB, PdxH, or Dxs activity as compared to the parent micro. In one embodiment, increased Epd, PdxA, PdxJ, PdxF, PdxB, PdxH, or Dxs activity is due to increased expression of a nucleic acid molecule encoding an Epd, PdxA, PdxJ, PdxF, PdxB, PdxH, and/or a Dxs polypeptide as compared to an unmodified parent organism. In another embodiment, increased epd, pdxA, pdxJ, pdxF, pdxB, pdxH, and/or dxs nucleic acid molecule expression is due to the introduction of nucleic acid molecules encoding an Epd, PdxA, PdxJ, PdxF, PdxB, PdxH, and/or a Dxs polypeptide into the organism.

The production methods of the present invention further can include recovering the B6 vitamer.

The instant invention also features genetically modified organisms, e.g., microorganisms, (i.e., organisms that contain one or more modifications or mutations in the genome) that are capable of producing significantly more of a B6 vitamer than an unmodified parent organism. In particular, this invention features microorganisms (including, for example, but not limited to bacteria, yeasts, fingi, and algae) or macro-organisms such as plants that, when genetically modified, produce an increased amount, e.g., at least about 10-fold more of a B6 vitamer, than the unmodified parent organism. Specific examples are given herein in which Bacillus subtilis and Escherichia coli strains have been genetically modified such that they produce significant amounts of a B6 vitamer. Accordingly, the present invention features organisms that have been genetically modified to increase the activity of one or more enzymes that catalyze(s) a step in the biosynthesis of a B6 vitamer, such that B6 vitamer production from said modified organism is increased compared to B6 production in an unmodified parent organism.

Yet another aspect of the invention features recombinant organisms, e.g., microorganisms which overexpress at least one Bacillus (e.g., B. subtilis) B6 vitamer biosynthetic enzyme (e.g., at least one of the yaaD, or yaaE gene products) or at least one of the epd, pdxA, pdxJ, pdxF, pdxB, pdxH, and/or dxs gene products, are described. In one embodiment, the recombinant microorganism is Gram positive (e.g., microorganisms belonging to the genus Bacillus, Cornyebacterium, Lactobacillus, Lactococci or Streptomyces). In another embodiment, the recombinant microorganism is Gram negative. Particularly preferred is a Bacillus recombinant microorganism (e.g., Bacillus licheniformis, Bacillus amyloliquefaciens, Bacillus subtilis, Bacillus pumilus, Bacillus halodurans, and the like).

Recombinant vectors that contain genes encoding Bacillus B6 vitamer biosynthetic enzymes, e.g., yaaD or yaaE genes, or homologues thereof, or epd, pdxA, pdxJ, pdxF, pdxB, pdxH, or dxs genes, or homologues thereof, are also described.

Other features and advantages of the invention will be apparent from the following detailed description and claims.

### BRIEF DESCRIPTION OF THE DRAWINGS

FIG. 1 depicts the chemical structures of vitamin B6 and related compounds.

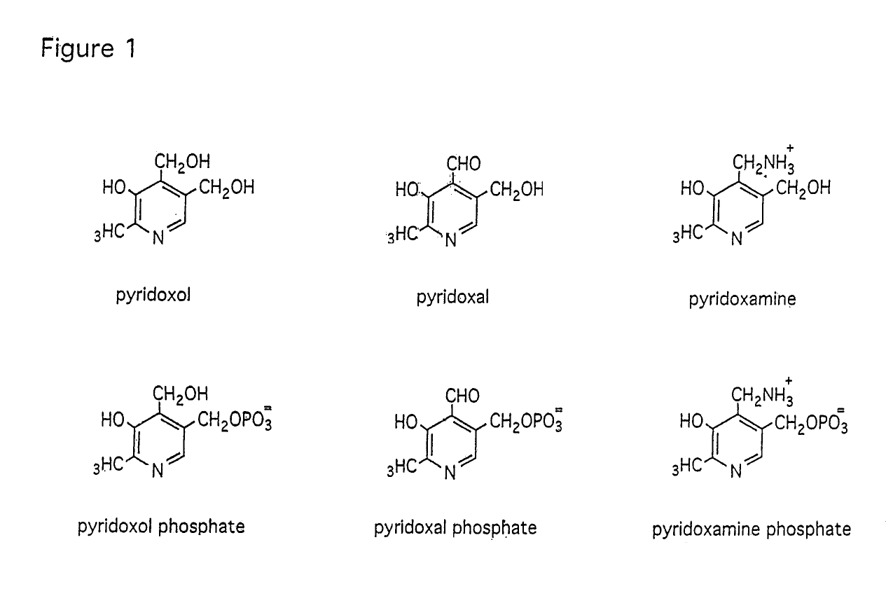

FIG. 2 depicts the biosynthetic pathway for PLP in E. coli.

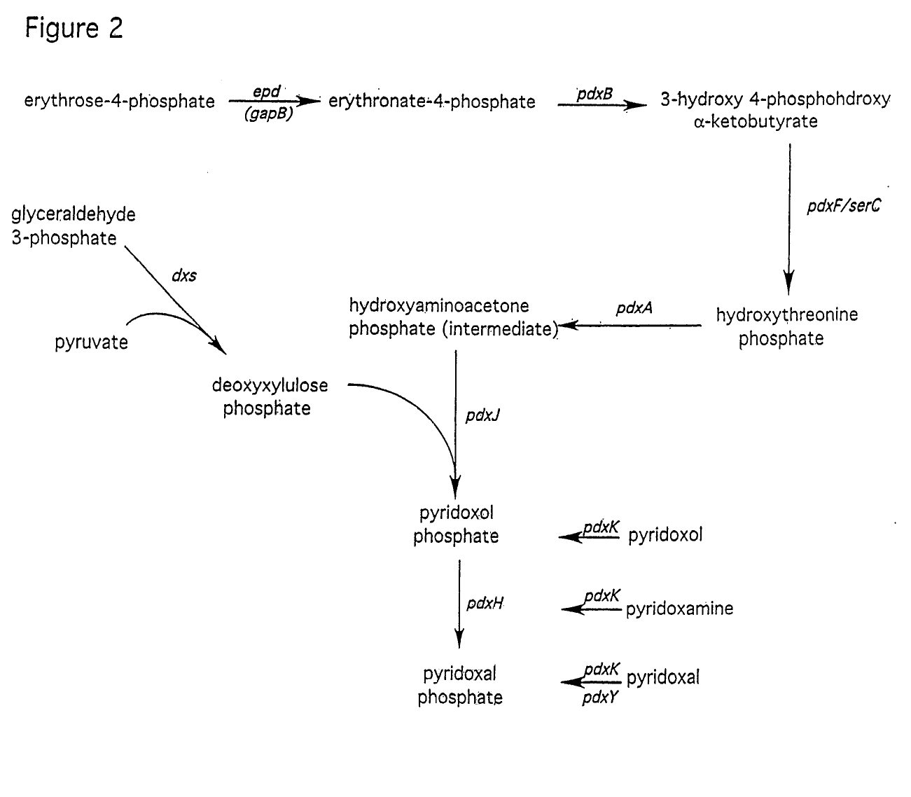

FIG. 3 depicts the standard curves generated by Saccharomyces uarum strain ATCC 9080 after feeding serial dilutions of PN, PL, and PM (as described in Example 1).

FIG. 4 is a schematic representation of the plasmid pDX1F.

FIG. 5 is a schematic representation of the plasmid pDX11F.

FIG. 6 is a schematic representation of the plasmid pDX14R.

FIG. 7 is a schematic representation of the plasmid pDX17R.

### DETAILED DESCRIPTION OF THE INVENTION

The present invention is based, at least in part, on the identification of Bacillus (e.g., B. subtilis) genes that encode essential enzymes of the B6 vitamer biosynthetic pathway. In particular, the present invention features methods based on manipulation of the B6 vitamer biosynthetic pathway in an organism, e.g. a microorganism such that certain desirable compounds are produced.

In particular, the invention is based, at least in part, on the discovery that the yaaD and yaaE genes of B. subtilis are required for B6 vitamer synthesis, including, but not limited to, pyridoxine (or pyridoxol (PN)), pyridoxal (PL), pyridoxamine (PM), or the 5′ phosphorylated derivatives of any of the three aforementioned compounds (PNP, PLP, and PMP). The yaaD and yaaE genes are adjacent on an operon, e.g., the yaaDE operon. The yaaD and yaaE genes encode the YaaD and YaaE proteins, respectively. Accordingly, based on the discovery of the activity of the polypeptides encoded by the yaaD and yaaE genes, or homologues thereof; in B6 vitamer synthesis, the invention is also based, at least in part, on the discovery that modulation of YaaD and/or YaaE activity results in the modulation of B6 vitamer production.

Accordingly, in one aspect, the invention includes a method for producing a B6 vitamer comprising culturing an organism with an increased YaaD and/or YaaE activity (e.g., YaaD and YaaE activity or YaaD activity) as compared to the parent organism. In one embodiment, increased YaaD and/or YaaE activity (e.g. YaaD and YaaE activity or YaaD activity) is due to increased expression of a nucleic acid molecule encoding a YaaD polypeptide and/or a YaaE polypeptide (e.g., a YaaD polypeptide and a YaaE polypeptide or a YaaD polypeptide) as compared to an unmodified parent organism. In another embodiment, increased yaaD and/or yaaE nucleic acid molecule expression (e.g., yaaD and yaaE nucleic acid expression or yaaD nucleic acid expression) is due to the introduction of nucleic acid molecules encoding a YaaD polypeptide and/or a YaaE polypeptide (e.g., a YaaD polypeptide and a YaaE polypeptide or a YaaD polypeptide) into the organism. In still another embodiment, the yaaD nucleic acid encodes a polypeptide comprising the amino acid sequence of SEQ ID NO:21 and/or the yaaE nucleic acid encodes a polypeptide comprising the amino acid sequence SEQ ID NO:23. In still another embodiment, a yaaD nucleic acid encodes a polypeptide comprising an amino acid sequence which is at least 30% identical to the amino acid sequence of SEQ ID NO:21, the polypeptide having a YaaD activity. In still another embodiment, a yaaE nucleic acid encodes a polypeptide comprising an amino acid sequence which is at least 30% identical to the amino acid sequence of SEQ ID NO:23, the polypeptide having a YaaE activity. In a further embodiment, the nucleic acid molecule comprising the nucleotide sequence of SEQ ID NO:20 and/or SEQ ID NO:22 is introduced. The nucleotide sequence of yaaD is set forth as SEQ ID NO:20 and the nucleotide sequence of yaaE is set forth as SEQ ID NO:22.

In another aspect, the invention provides methods for producing a B6 vitamer comprising culturing an organism with an increased Epd, PdxA, PdxJ, PdxF, PdxB, PdxH, and/or Dxs activity as compared to the parent organism. In one embodiment, increased Epd, PdxA, PdxJ, PdxF, PdxB, PdxH, and/or Dxs activity is due to increased expression of a nucleic acid molecule encoding an Epd, PdxA, PdxJ, PdxF, PdxB, PdxH, and/or a Dxs polypeptide as compared to an unmodified parent organism. In another embodiment, increased epd, pdxA, pdxJ, pdxF, pdxB, pdxH, and/or dxs nucleic acid molecule expression is due to the introduction of nucleic acid molecules encoding an Epd, PdxA, PdxJ, PdxF, PdxB, PdxH, and/or a Dxs polypeptide into the organism. In still another embodiment, the pdxA nucleic acid encodes a polypeptide comprising the amino acid sequence of SEQ ID NO:25 and/or wherein said pdxJ nucleic acid encodes a polypeptide comprising the amino acid sequence SEQ ID NO:27. In a further embodiment, the pdxA nucleic acid encodes a polypeptide comprising an amino acid sequence which is at least 30% identical to the amino acid sequence of SEQ ID NO:25, the polypeptide having a PdxA activity. In still another embodiment, the pdxJ nucleic acid encodes a polypeptide comprising an amino acid sequence which is at least 30% identical to the amino acid sequence of SEQ ID NO:27, and having a PdxJ activity. In yet another embodiment, the nucleic acid molecule comprising the nucleotide sequence of SEQ ID NO:24 and/or SEQ ID NO:26 is introduced. The nucleotide sequence of pdxA is set forth as SEQ ID NO:24. The nucleotide sequence of pdxJ is set forth as SEQ ID NO:26.

Overexpression of the yaaDE operon with a strong constitutive promoter or the deregulation of the expression of the yaaD or yaaE gene(s) leads to significantly increased production of B6 vitamers. These quantities are significantly higher relative to the associated parent strains than those reported in previous studies, which have employed mutant E. coli strains (Dempsey and Arcement (1971) J. Bacteriol. 107 (2): 580-582), or mutant B. subtilis strains (Pflug, W., and Lingens, F., (1978) Hoppe-Seyler's Z. Physiol. Chem. 359: 559-570).

Accordingly, the present invention features organisms, e.g., microorganisms that have been genetically modified to increase the activity of one or more enzymes that catalyze a step in the biosynthesis of a B6 vitamer, such that B6 vitamer production from the modified organism is increased compared to B6 production in an unmodified parent organism. In one embodiment, B6 vitamer production is at least ten-fold higher than from the unmodified parent organism. In another embodiment, the organism is genetically modified to overexpress one or more genes that encodes an enzyme that catalyzes a step in the biosynthesis of a B6 vitamer, e.g. yaaD or yaaE, or a homologue thereof, or epd, pdxA, pdxJ, pdxF, pdxB, pdxH, or dxs, or a homologue thereof. The organism, e.g., microorganism, may be, for example, B. subtilis or E. coli.

The present invention also features methods of producing a B6 vitamer comprising culturing an organism, e.g., a microorganism that has been genetically modified to overexpress one or more genes that encodes an enzyme that catalyzes a step in the biosynthesis of a B6 vitamer, such that B6 vitamer production from said modified organism is increased compared to B6 production in an unmodified parent organism, under conditions such that the B6 vitamer is produced. The B6 vitamer may then be subsequently recovered.

The terms “B6 vitamer” or “B6 vitamers,” as used herein, shall refer to any compound or mixture of compounds that has any biological activity in any biological assay for vitamin B6. B6 vitamers include, but are not limited to, pyridoxine (also called pyridoxol or PN), pyridoxal (PL), pyridoxamine (PM), the 5′ phosphorylated derivatives of any of the three aforementioned compounds (PNP, PLP, and PMP), and any derivative or related compound that can be converted to the active forms (PLP or PMP) in a test organism. Thus, for example, the acetate esters or other esters of any of the available hydroxyl groups of any of the aforementioned six compounds, and which are likely to be hydrolyzed by specific or non-specific esterases, are included in B₆ vitamers. Also, various salts, such as hydrochloride salts, of any of the aforementioned compounds are included in B6 vitamers.

The term “B6 vitamer biosynthetic pathway” includes the biosynthetic pathway involving B6 vitamer biosynthetic enzymes (e.g., polypeptides encoded by biosynthetic enzyme-encoding genes), compounds (e.g., precursors, substrates, intermediates or products), cofactors and the like utilized in the formation or synthesis of B6 vitamers. The term “B6 vitamer biosynthetic pathway” includes the biosynthetic pathway leading to the synthesis of B6 vitamers in a microorganism (e.g. in vivo) as well as the biosynthetic pathway leading to the synthesis of B6 vitamers in vitro.

The term “B6 vitamer biosynthetic enzyme” includes an enzyme which is involved in the synthesis of any B6 vitamer in an organism or microorganism, e.g., an enzyme that is rate limiting for B6 vitamer synthesis in said organism or microorganism. B6 vitamer biosynthetic enzymes include, for example, YaaD, YaaE, Epd, PdxA, PdxJ, PdxF, PdxB, PdxH, and/or Dxs, or homologues thereof.

The term “B6 vitamer biosynthetic enzyme activity” includes, for example, any enzyme activity which results in B6 vitamer synthesis. For example, PdxA activity includes the catalysis of hydroxythreonine to hydroxyaminoacetone, and PdxJ activity includes the catalysis of hydroxyaminoacetone and deoxyeylatose to pyridoxol phosphate.

Overproduction or increased activity of the rate limiting enzyme for B6 vitamer production in any organism that is capable of producing B6 vitamers will lead to overproduction of B6 vitamers. For example, overexpression or increased activity of any of the E. coli genes involved in the PLP pathway will give a measurable increase ir B6 vitamer production. Specifically, overexpression or increased activity of the E. coli genes epd, pdxA, pdxJ, pdxF, pdxB, pdxH, or dxs, homologues thereof, or a combination thereof, in E. coli may lead to an increase in B6 vitamer production. Moreover, overexpression or increased activity of genes of the type B pathway, e.g., the yaaD or yaaE genes (e.g., the yaaD and yaaE genes or the yaaD gene) will result in increased B6 vitamer production in host organisms of the type A pathway, e.g., E. coli. Likewise, overexpression or increased activity of genes of the type A pathway, e.g., the epd, pdxA, pdxJ, pdxF, pdxB, pdxH, or dxs genes will result in increased B6 vitamer production in host organisms of the type B pathway, e.g., B. subtilis.

A “biological assay for a B6 vitamer” includes, for example, any assay that is capable of quantifying B6 vitamer activity by measuring growth of an organism that requires the feeding of a B6 vitamer (i.e., a compound that the fed organism can convert into PLP or PMP) for growth. Samples to be assayed are diluted serially in an appropriate medium and fed to the appropriate organism. Standard curves are generated by serially diluting known amounts of PL, PN, or PM, and feeding these dilutions to the test organism. By comparing dilutions of the unknown samples to the standard curves, total B6 vitamer activity can be determined, for example as PL equivalents if PL was used to generate the standard curve.

Various aspects of the invention are described in further detail in the following subsections.

I. Genes Encoding Various B6 Vitamer Biosynthetic Enzymes

In one embodiment, the present invention features targeting or modifying various biosynthetic genes or enzymes of the B6 vitamer biosynthetic pathway. In particular, the invention features modifying various enzymatic activities associated with said pathways by modifying or altering the genes encoding said biosynthetic enzymes.

The term “gene”, as used herein, includes a nucleic acid molecule (e.g., a DNA molecule or segment thereof) that, in an organism, can be separated from another gene or other genes, by intergenic DNA (i.e., intervening or spacer DNA which naturally flanks the gene and/or separates genes in the chromosomal DNA of the organism). Alternatively, a gene may slightly overlap another gene (e.g., the 3′ end of a first gene overlapping the 5′ end of a second gene), said overlapping genes separated from other genes by intergenic DNA. A gene may direct synthesis of an enzyme or other protein molecule (e.g., may comprise coding sequences, for example, a contiguous open reading frame (ORF) which encodes a protein) or may itself be functional in the organism. A gene in an organism, may be clustered in an operon, as defined herein, said operon being separated from other genes and/or operons by the intergenic DNA. An “isolated gene”, as used herein, includes a gene which is essentially free of sequences which naturally flank the gene in the chromosomal DNA of the organism from which the gene is derived (i.e., is free of adjacent coding sequences which encode a second or distinct protein, adjacent structural sequences or the like) and optionally includes 5′ and 3′ regulatory sequences, for example promoter sequences and/or terminator sequences. In one embodiment, an isolated gene includes predominantly coding sequences for a protein (e.g., sequences which encode Bacillus proteins). In another embodiment, an isolated gene includes coding sequences for a protein (e.g., for a Bacillus protein) and adjacent 5′ and/or 3′ regulatory sequences from the chromosomal DNA of the organism from which the gene is derived (e.g., adjacent 5′ and/or 3′ Bacillus regulatory sequences). Preferably, an isolated gene contains less than about 10 kb, 5 kb, 2 kb, 1 kb, 0.5 kb, 0.2 kb, 0.1 kb, 50 bp, 25 bp or 10 bp of nucleotide sequences that naturally flank the gene in the chromosomal DNA of the organism from which the gene is derived.

The term “operon” includes at least two adjacent genes or ORFs, optionally overlapping in sequence at either the 5′ or 3′ end of at least one gene or ORF. The term “operon” includes a coordinated unit of gene expression that contains a promoter and possibly a regulatory element associated with one or more adjacent genes or ORFs (e.g., structural genes encoding enzymes, for example, biosynthetic enzymes). Expression of the genes (e.g., structural genes) can be coordinately regulated, for example, by regulatory proteins binding to the regulatory element or by anti-termination of transcription. The genes of an operon (e.g., structural genes) can be transcribed to give a single mRNA that encodes all of the proteins.

A “gene having a mutation” or “mutant gene” as used herein, includes a gene having a nucleotide sequence which includes at least one alteration (e.g., substitution, insertion, deletion) such that the polypeptide or protein encoded by said mutant exhibits an activity that differs from the polypeptide or protein encoded by the wild-type nucleic acid molecule or gene. In one embodiment, a gene having a mutation or mutant gene encodes a polypeptide or protein having an increased activity as compared to the polypeptide or protein encoded by the wild-type gene, for example, when assayed under similar conditions (e.g., assayed in microorganisms cultured at the same temperature). As used herein, an “increased activity” or “increased enzymatic activity” is one that is at least 5% greater than that of the polypeptide or protein encoded by the wild-type nucleic acid molecule or gene, preferably at least 5-10% greater, more preferably at least 10-25% greater and even more preferably at least 25-50%, 50-75% or 75-100% greater than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, an “increased activity” or “increased enzymatic activity” can also include an activity that is at least 1.25-fold greater than the activity of the polypeptide or protein encoded by the wild-type gene, preferably at least 1.5-fold greater, more preferably at least 2-fold greater and even more preferably at least 3-fold, 4-fold, 5-fold, 10-fold, 20-fold, 50-fold, 100-fold or greater than the activity of the polypeptide or protein encoded by the wild-type gene.

In another embodiment, a gene having a mutation or mutant gene encodes a polypeptide or protein having a reduced activity as compared to the polypeptide or protein encoded by the wild-type gene, for example, when assayed under similar conditions (e.g., assayed in microorganisms cultured at the same temperature). A mutant gene also can encode no polypeptide or have a reduced level of production of the wild-type polypeptide. As used herein, a “reduced activity” or “reduced enzymatic activity” is one that is at least 5% less than that of the polypeptide or protein encoded by the wild-type nucleic acid molecule or gene, preferably at least 5-10% less, more preferably at least 10-25% less and even more preferably at least 25-50%, 50-75% or 75-100% less than that of the polypeptide or protein encoded by the wild-type nucleic acid molecule or gene. Ranges intermediate to the above-recited values, e.g. 75-85%, 85-90%, 90-95%, are also intended to be encompassed by the present invention. As used herein, a “reduced activity” or “reduced enzymatic activity” can also include an activity that has been deleted or “knocked out” (e.g., approximately 100% less activity than that of the polypeptide or protein encoded by the wild-type nucleic acid molecule or gene).

Activity can be determined according to any well accepted assay for measuring activity of a particular protein of interest. Activity can be measured or assayed directly, for example, measuring an enzymatic or biological activity of a protein isolated or purified from a cell or microorganism. Alternatively, an activity can be measured or assayed within a cell or microorganism or in an extracellular medium. For example, assaying for a mutant gene (i.e., said mutant encoding a reduced enzymatic activity) can be accomplished by expressing the mutated gene in a microorganism, for example, a mutant microorganism in which the enzyme is temperature-sensitive, and assaying the mutant gene for the ability to complement a temperature sensitive (Ts) mutant for enzymatic activity. A mutant gene that encodes an “increased enzymatic activity” can be one that complements the Ts mutant more effectively than, for example, a corresponding wild-type gene. A mutant gene that encodes a “reduced enzymatic activity” is one that complements the Ts mutant less effectively than, for example, a corresponding wild-type gene.

It will be appreciated by the skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant gene (e.g., encoding a mutant polypeptide or protein), as defined herein, is readily distinguishable from a nucleic acid or gene encoding a protein homologue in that a mutant gene encodes a protein or polypeptide having an altered activity, optionally observable as a different or distinct phenotype in an organism expressing said mutant gene or producing said mutant protein or polypeptide (i.e., a mutant microorganism) as compared to a corresponding organism expressing the wild-type gene. By contrast, a protein homologue has an identical or substantially similar activity, optionally phenotypically indiscernible when produced in an organism, e.g., a microorganism, as compared to a corresponding organism, e.g., microorganism, expressing the wild-type gene. Accordingly it is not, for example, the degree of sequence identity between nucleic acid molecules, genes, protein or polypeptides that serves to distinguish between homologues and mutants, rather it is the activity of the encoded protein or polypeptide that distinguishes between homologues and mutants: homologues having, for example, low (e.g., 30-50% sequence identity) sequence identity yet having substantially equivalent functional activities, and mutants, for example sharing 99% sequence identity yet having dramatically different or altered functional activities.

As used herein, the term “homologue”, e.g., a yaaD, yaaE, epd, pdxA, pdxJ, pdxF, pdxB, pdxH, or dxs homologue, refers to a molecule having at least about 30%, 35%, 40%, 45%, 50%, 55%, 60%, 65%, 70%, 75%, 80%, 85%, 90%, 95%, 96%, 97%, 98%, 99%, or more nucleotide or amino acid sequence identity to a yaaD, yaaE, epd, pdxA, pdxJ, pdxF, pdxB, pdxH, or dxs nucleic acid or protein molecule and having a specific functional activity, e.g., a yaaD activity, a yaaE activity, a pdxA activity, or a pdxJ activity. A yaaD activity, a yaaE activity, a pdxA activity, or a pdxJ activity includes the ability of the homologue to rescue an auxotroph, e.g., an organism which is auxotrophic due to the mutation or deletion of a gene encoding the relevant enzyme involved in a B6 biosynthetic pathway, in a test system, or the ability of the homologue to produce a desired product, e.g., a B6 vitamer or intermediate in the B6 vitamer biosynthetic pathway.

In one embodiment, methods for testing potential homologues, e.g., yaaD, yaaE, epd, pdxA, pdxJ, pdxF, pdxB, pdxH, or &cs homologues, for a yaaD, yaaE, epd, pdxA, pdxJ, pdxF, pdxB, pdxH, or dxs functional activity include complementation assays. For example, a complementation assay includes an assay wherein the potential homologue, e.g., a potential homologue isolated from a plant organism or a microorganism, is expressed in an auxotrophic Bacillus microorganism in which a portion of the yaaD or yaaE gene has been deleted, and the ability of the potential homologue to rescue the microorganism from auxotrophy is measured. In one embodiment, a Bacillus promoter and ribosome binding site, such as, for example, a ribosome binding site described herein, is utilized for the expression of the homologue in the Bacillus microorganism. In another embodiment, the potential homologue is expressed in an auxotrophic E. coli microorganism (for example, a pdxA, pdxB, pdxF, pdxJ, dxs, or epd mutant, and the ability of the potential homologue to rescue the microorganism from auxotrophy is measured.

A secondary assay for testing potential homologues, e.g., yaaD, yaaE, epd, pdxA, pdxJ, pdxF, pdxB, pdxH, or dxs homologues, for a yaaD, yaaE, epd, pdxA, pdxJ, pdxF, pdxB, pdxH, or dxs functional activity includes an assay which directly measures the amount of desired product, e.g., a B6 vitamer, produced by the recombinant organism. For example, the amount of B6 vitamer produced may be measured by methods described herein, e.g., by HPLC analysis, and by other methods known in the art.

A “potential homologue” as used herein, includes a molecule with 30%, 35%, 40%, 45%, 50%, 55%, 60%, 65%, 70%, 75%, 80%, 85%, 90%, 95%, 96%, 97%, 98%, 99%, or more nucleotide or amino acid sequence identity to a yaaD, yaaE, epd, pdxA, pdxJ, pdxF, pdxB, pdxH, or dxs nucleic acid or protein molecule, where a functional activity of the molecule has not yet been established through, for example, an assay described herein for measuring functional activity.

In a preferred embodiment, the genes of the present invention are derived from Bacillus. The term “derived from Bacillus” or “Bacillus-derived” includes a gene which is naturally found in microorganisms of the genus Bacillus. In another preferred embodiment, the genes of the present invention are derived from a microorganism selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the gene is derived from Bacillus brevis or Bacillus stearothermophilus. In another preferred embodiment, the genes of the present invention are derived from a microorganism selected from the group consisting of Bacillus licheniformis, Bacillus amyloliquefaciens, Bacillus subtilis, and Bacillus pumilus. In a particularly preferred embodiment, the gene is derived from Bacillus subtilis (e.g., is Bacillus subtilis-derived). The term “derived from Bacillus subtilis” or “Bacillus subtilis-derived” includes a gene which is naturally found in the microorganism Bacillus subtilis. Included within the scope of the present invention are Bacillus-derived genes (e.g., B. subtilis-derived genes), for example, Bacillus or B. subtilis yaaD or yaaE genes.

In a preferred embodiment, the genes of the present invention are derived from Escherichia. The term “derived from Escherichia” or “Escherichia-derived” includes a gene which is naturally found in microorganisms of the genus Escherichia. In a particularly preferred embodiment, the gene is derived from Escherichia coli (e.g., is Escherichia coli-derived). The term “derived from Escherichia coli” or “Escherichia coli-derived” includes a gene which is naturally found in the microorganism Escherichia coli. Included within the scope of the present invention are Escherichia-derived genes (e.g., Escherichia coli-derived genes), for example, Escherichia or Escherichia coli epd, pdxA, pdxJ, pdxF, pdxB, pdxH, or dxs genes.

In another preferred embodiment, the genes of the present invention are derived, for example, from any one of the organisms or microorganisms listed in Section III or in either of Tables 9 or 10 (or in the GenBank records referred to by Accession Nos. listed therein).

II Recombinant Nucleic Acid Molecules and Vectors

The present invention further features recombinant nucleic acid molecules (e.g., recombinant DNA molecules) that include genes described herein (e.g., isolated genes), preferably Bacillus genes, more preferably Bacillus subtilis genes, even more preferably Bacillus subtilis B6 vitamer biosynthetic genes. The term “recombinant nucleic acid molecule” includes an isolated nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). Preferably, a recombinant nucleic acid molecule (e.g., a recombinant DNA molecule) includes an isolated gene of the present invention operably linked to regulatory sequences. The phrase “operably linked to regulatory sequence(s)” means that the nucleotide sequence of the gene of interest is linked to the regulatory sequence(s) in a manner which allows for expression (eg., enhanced, increased, constitutive, basal, attenuated, decreased or repressed expression) of the gene, preferably expression of a gene product encoded by the gene (e.g., when the recombinant nucleic acid molecule is included in a recombinant vector, as defined herein, and is introduced into a microorganism). A “recombinant organism” is any organism that contains a recombinant nucleic acid molecule.

The term “regulatory sequence” includes nucleic acid sequences that affect (e.g., modulate or regulate) expression of other nucleic acid sequences (i.e., genes). In one embodiment, a regulatory sequence is included in a recombinant nucleic acid molecule in a similar or identical position and/or orientation relative to a particular gene of interest as is observed for the regulatory sequence and gene of interest as it appears in nature, e.g., in a native position and/or orientation. For example, a gene of interest can be included in a recombinant nucleic acid molecule operably linked to a regulatory sequence which accompanies or is adjacent to the gene of interest in the natural organism (e.g., operably linked to “native” regulatory sequences (e.g., to the “native” promoter). Alternatively, a gene of interest can be included in a recombinant nucleic acid molecule operably linked to a regulatory sequence which accompanies or is adjacent to another (e.g., a different) gene in the natural organism. Alternatively, a gene of interest can be included in a recombinant nucleic acid molecule operably linked to a regulatory sequence from another organism. For example, regulatory sequences from other microbes (e.g., other bacterial regulatory sequences, bacteriophage regulatory sequences and the like) can be operably linked to a particular gene of interest.

In one embodiment, a regulatory sequence is a non-native or non-naturally-occurring sequence (e.g., a sequence which has been modified, mutated, substituted, derivatized, deleted including sequences which are chemically synthesized). Preferred regulatory sequences include promoters, enhancers, termination signals, anti-termination signals, ribosome binding sites and other expression control elements (e.g., sequences to which repressors or inducers bind and/or binding sites for transcriptional and/or translational regulatory proteins, for example, in the transcribed mRNA). Such regulatory sequences are described, for example, in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Regulatory sequences include those which direct constitutive expression of a nucleotide sequence in a microorganism (e.g., constitutive promoters and strong constitutive promoters), those which direct inducible expression of a nucleotide sequence in a microorganism (e.g., inducible promoters, for example, xylose inducible promoters) and those which attenuate or repress expression of a nucleotide sequence in a microorganism (e.g., attenuation signals or repressor sequences). It is also within the scope of the present invention to regulate expression of a gene of interest by removing or deleting regulatory sequences. For example, sequences involved in the negative regulation of transcription can be removed such that expression of a gene of interest is enhanced.

In one embodiment, a recombinant nucleic acid molecule of the present invention includes a nucleic acid sequence or gene that encodes at least one bacterial gene product (e.g., a B6 vitamer biosynthetic enzyme, e.g., the gene product of yaaD and/or yaaE, (e.g., the gene product of yaaD and yaaE or yaaD) or a homologue thereof) operably linked to a promoter or promoter sequence. Preferred promoters of the present invention include Bacillus promoters and/or bacteriophage promoters (e.g., bacteriophage which infect Bacillus). In one embodiment, a promoter is a Bacillus promoter, preferably a strong Bacillus promoter (e.g., a promoter associated with a biochemical housekeeping gene in Bacillus or a promoter associated with a glycolytic pathway gene in Bacillus). In another embodiment, a promoter is a bacteriophage promoter. In a preferred embodiment, the promoter is from the bacteriophage SP01. In a particularly preferred embodiment, a promoter is selected from the group consisting of P₁₅, P₂₆, or P_{veg} having for example, the following respective sequences: GCTATTGACGACAGCTATGGTTCACTGTCCACCAACCAAAACTGTGCTCAGT ACCGCCAATATTTCTCCCTTGAGGGGTACAAAGAGGTGTCCCTAGAAGAGAT CCACGCTGTGTAAAAATTTTACAAAAAGGTATTGACTTTCCCTACAGGGTGT GTAATAATTTAATTACAGGCGGGGGCAACCCCGCCTGT (SEQ ID NO:9), GCCTACCTAGCTTCCAAGAAAGATATCCTAACAGCACAAGAGCGGAAAGAT GTTTTGTTCTACATCCAGAACAACCTCTGCTAAAATTCCTGAAAAATTTTGC AAAAAGTTGTTGACTTTATCTACAAGGTGTGGTATAATAATCTTAACAACAG CAGGACGC (SEQ ID NO:10); and GAGGAATCATAGAATTTTGTCAAAATAATITTTTATTGACAACGTCTTATTAAC GTTGATATAATTTAAATTTTATTTGACAAAAATGGGCTCGTGTTGTACAATA AATGTAGTGAGGTGGATGCAATG (SEQ ID NO:11). Additional preferred promoters include tef (the translational elongation factor (TEF) promoter) and pyc (the pyruvate carboxylase (PYC) promoter), which promote high level expression in Bacillus (e.g., Bacillus subtilis). Additional preferred promoters, for example, for use in Gram positive microorganisms include, but are not limited to, amy and SPO2 promoters. Additional preferred promoters, for example, for use in Gram negative microorganisms include, but are not limited to, tac, trp, tet, trp-tet, lpp, lac, lpp-lac, lacIQ, T7, T5, T3, gal, trc, ara, SP6, λ-PR or λ-PL.

In another embodiment, a recombinant nucleic acid molecule of the present invention includes a terminator sequence or terminator sequences (e.g., transcription terminator sequences). The term “terminator sequences” includes regulatory sequences that serve to terminate transcription of mRNA. Terminator sequences (or tandem transcription terminators) can further serve to stabilize mRNA (e.g., by adding structure to mRNA), for example, against nucleases.

In yet another embodiment, a recombinant nucleic acid molecule of the present invention includes sequences which allow for detection of the vector containing said sequences (i.e., detectable and/or selectable markers), for example, genes that encode antibiotic resistance or sequences that overcome auxotrophic mutations, for example, trpC, fluorescent markers, drug markers, and/or colorimetric markers (e.g. lacZ/β-galactosidase). In yet another embodiment, a recombinant nucleic acid molecule of the present invention includes an artificial ribosome binding site (RBS) or a sequence that becomes transcribed into an artificial RBS. The term “artificial ribosome binding site (RBS)” includes a site within an mRNA molecule (e.g., coded within DNA) to which a ribosome binds (e.g., to initiate translation) which differs from a native RBS (e.g., a RBS found in a naturally-occurring gene) by at least one nucleotide. Preferred artificial RBSs include about 5-6, 7-8, 9-10, 11-12, 13-14, 15-16, 17-18, 19-20, 21-22, 23-24, 25-26, 27-28, 29-30 or more nucleotides of which about 1-2, 3-4, 5-6, 7-8, 9-10, 11-12, 13-15 or more differ from the native RBS (e.g., the native RBS of a gene of interest, for example, the native yaaD RBS GAAATCATATAACTATACCTTGATTAGGGGGACCAAGAAATG (SEQ ID NO:12) or the native yaaE RBS CAAGAACGCGGCTGGTAAGAACATAGGAGCGCTGCTGACATG (SEQ ID NO:13)).

Preferably, nucleotides that differ are substituted such that they are identical to one or more nucleotides of an ideal RBS when optimally aligned for comparisons. Artificial RBSs can be used to replace the naturally-occurring or native RBSs associated with a particular gene. Artificial RBSs preferably increase translation of a particular gene. Preferred artificial RBSs (e.g., RBSs for increasing the translation of yaaE, for example, of B. subtilis) are set forth in Table 1, below.

TABLE 1
Preferred Ribosome Binding Sites

| 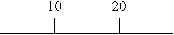 | | SEQ ID NO: |
|---|---|---|
| Native_yaaD | ---GAAATCATATAACTATACCTTGATTAGGGGGACC-AAGAAATG | 12 |
| Native_yaaE | CAAGAACGCGGCTGGTAAGAACAT----AGGAGCGCTGCTGACATG | 13 |
| IDEAL_RES | --------------TCTAGAAAGG----AGGTG-----A------- | 14 |
| RBS1 | --------------TCTAGAAGG-----AGGAG-----AAAACATG | 15 |
| RBS2 | --------------TCTAGAGG------AGGAG-----AAAACATG | 16 |
| RES101 | ---------------TAAGAACAA----AGGAGGAGAGCTGACATG | 17 |
| RBS103 | ---------------TAAGAAGAA----AGGAGGTGAGCTGACATG | 18 |
| RBS102 | ---------------TAAGAACAG----AGGAGGAGAGCTGACATG | 19 |

The present invention further features vectors (e.g. recombinant vectors) that include nucleic acid molecules (e.g., genes or recombinant nucleic acid molecules comprising said genes) as described herein. The term “recombinant vector” includes a vector (e.g., plasmid, phage, phasmid, virus, cosmid or other purified nucleic acid vector) that has been altered, modified or engineered such that it contains greater, fewer or different nucleic acid sequences than those included in the native or natural nucleic acid molecule from which the recombinant vector was derived. Preferably, the recombinant vector includes a biosynthetic enzyme-encoding gene or recombinant nucleic acid molecule including said gene, operably linked to regulatory sequences, for example, promoter sequences, terminator sequences and/or artificial ribosome binding sites (RBSs), as defined herein. In another embodiment, a recombinant vector of the present invention includes sequences that enhance replication in bacteria (e.g., replication-enhancing sequences). In one embodiment, replication-enhancing sequences are derived from E. coli. In another embodiment, replication-enhancing sequences are derived from pBR322. In another embodiment, replication-enhancing sequences are derived from pSC101.

In yet another embodiment, a recombinant vector of the present invention includes antibiotic resistance sequences. The term “antibiotic resistance sequences” includes sequences which promote or confer resistance to antibiotics on the host organism (e.g., Bacillus). In one embodiment, the antibiotic resistance sequences are selected from the group consisting of cat (chloramphenicol resistance) sequences, tet (tetracycline resistance) sequences, erm (erythromycin resistance) sequences, neo (neomycin resistance) sequences, kan (kanamycin resistance) and spec (spectinomycin resistance) sequences. Recombinant vectors of the present invention can further include homologous recombination sequences (e.g., sequences designed to allow recombination of the gene of interest into the chromosome of the host organism). For example, bpr, vpr, and/or amyE sequences can be used as homology targets for recombination into the host chromosome. It will further be appreciated by one of skill in the art that the design of a vector can be tailored depending on such factors as the choice of microorganism to be genetically engineered, the level of expression of gene product desired and the like.

III. Recombinant Organisms

The present invention further features organisms, i.e., recombinant organisms e.g., recombinant microorganisms, that include vectors or genes (e.g., wild-type and/or mutated genes) as described herein. As used herein, the term “recombinant organism” includes an organism or a microorganism (e.g., bacteria, yeast cell, fungal cell, plant cell, etc.) that has been genetically altered, modified or engineered (e.g., genetically engineered) such that it exhibits an altered, modified or different genotype and/or phenotype (e.g., when the genetic modification affects coding nucleic acid sequences of the organism) as compared to the naturally-occurring organism from which it was derived, e.g., a wild type or unmodified organism or parent organism or host organism.

Possible host organisms include, for example, plants, algae, fingi, yeasts, and other organisms and microorganisms. The plant may be a monocot, dicot or gymnosperm. Preferred microbes are those used in agriculture or by industry, and those that are pathogenic for plants or animals. Plants include arabidopsis; field crops (e.g., alfalfa, barley, bean, cereals, corn, cotton, flax, lucerne, hemp, millet, oats, pea, rape, rice, rye, safflower, sorghum, soybean, sunflower, tobacco, triticale, and wheat); vegetable crops (e.g., asparagus, beet, broccoli, cabbages, capsicum, carrot, cauliflower, celery, cucumber, eggplant, lettuces, onion, pepper, potato, pumpkin, maize, radish, spinach, squash, taro, tomato, and zucchini); fruit and grapevine and nut species (e.g., almond, apple, apricot, banana, black-berry, blueberry, cacao, cherry, coconut, cranberry, date, faJoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); Brassicaceae such as oilseed, canola, sugarbeet, sugarcane; and ornamentals (e.g. alder, ash, aspen, azalea, birch, boxwood, camellia, carnation, chrysanthemum, elm, fir, ivy, jasmine, juniper, oak, palm, poplar, pine, redwood, rhododendron, rose, rubber, and yew).

Fungi which may be used as host organisms include organisms in both the mold and yeast morphologies. Possible fungal host organisms include the phylum Ascomycota, including, for example, the subphyla Neolectomycetes, Pezizomycotina, Pneumocystidomycetes, Saccharomycotina, Schizosaccharomycetes, Taphrinomycetes, mitosporic Ascomycota, and unclassified Ascomycota; the phylum Basidiomycota, including, for example, the subphyla Hymenomecetes, Urediniomycetes, Ustilaginomycetes, Mitosporic, Masidiomycota, and unclassified Basidiomycota; the phylum Chytridiomycota, including, for example, the subphyla, Blastocladiales, Chytridiales, Monoblepharidales, Neocallimasticales, Spizellomycetales, and unclassified Chytridiomycota; the phylum Glomeromycota, including, for example, the subphylum Glomeromycetes; the phylum Microsporidia, including, for example, the subphyla, Apansporoblastina, Endoreticulatus, Gurleyidae, Nadelsporidae, Ordosporidae, Pansporablastina, Pereziidae, Pseudonosematidae, Tetramicriidae, and unclassified Microsporidia; the phylum Zygomycota, including, for example, the subphyla, Trichomycetes, Zygomycetes, and unclassified Zygomycota and unclassified Fungi.

Algae which may be used as host organisms include, for example, green algae, red algae, yellow-green algae, Cryptomonads, haptophytes, golden algae, euglenids, diatoms, and brown algae.

Other organisms, e.g., microorganisms, which may be used as host organisms include Gram negative bacteria (e.g., a microorganisms which excludes basic dye, for example, crystal violet) and Gram positive bacteria (e.g. a microorganism which retains basic dye, for example, crystal violet, due to the presence of a Gram-positive wall surrounding the microorganism). Gram negative bacteria include, for example, Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae. Gram positive bacteria include, for example, Bacillaceae, Micrococcaceae, and Peptococcaceae.

In a preferred embodiment, the recombinant microorganism is a Gram positive microorganism belonging to a genus selected from the group consisting of Bacillus, Cornyebacterium, Lactobacillus, Lactococci and Streptomyces. In a more preferred embodiment, the recombinant microorganism is of the genus Bacillus. In another preferred embodiment, the recombinant microorganism is selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the recombinant microorganism is Bacillus brevis or Bacillus stearothermophilus. In another preferred embodiment, the recombinant microorganism is selected from the group consisting of Bacillus licheniformis, Bacillus amyloliquefaciens, Bacillus subtilis, and Bacillus pumilus.

In another preferred embodiment, the recombinant microorganism is a Gram negative microorganism belonging to a genus selected from the group consisting of Salmonella, Escherichia; Klebsiella, Serratia, and Proteus. In a more preferred embodiment, the recombinant microorganism is of the genus Escherichia. In an even more preferred embodiment, the recombinant microorganism is Escherichia coli. In another embodiment, the recombinant microorganism is Saccharomyces (e.g., S. cerevisiae).

A preferred “recombinant” organism, e.g., microorganism of the present invention is an organism, e.g., a microorganism having a deregulated B6 vitamer biosynthesis pathway or enzyme. The term “deregulated” or “deregulation” includes the alteration or modification of at least one gene in an organism, e.g., a microorganism that encodes an enzyme in a biosynthetic pathway, such that the level or activity of the biosynthetic enzyme in the organism, e.g., the microorganism is altered or modified. Preferably, at least one gene that encodes an enzyme in a biosynthetic pathway is altered or modified such that the gene product is enhanced or increased. The phrase “deregulated pathway” can also include a biosynthetic pathway in which more than one gene that encodes an enzyme in a biosynthetic pathway is altered or modified such that the level or activity of more than one biosynthetic enzyme is altered or modified. The ability to “deregulate” a pathway (e.g., to simultaneously deregulate more than one gene in a given biosynthetic pathway) in an organism, e.g., a microorganism in some cases arises from the particular phenomenon of organisms, e.g., microorganisms in which more than one enzyme (e.g., two or three biosynthetic enzymes) are encoded by genes occurring adjacent to one another on a contiguous piece of genetic material termed an “operon” (defined herein). Due to the coordinated regulation of genes included in an operon, alteration or modification of the single promoter and/or regulatory element can result in alteration or modification of the expression of more than one gene product encoded by the operon. Alteration or modification of the regulatory element can include, but is not limited to removing the endogenous promoter and/or regulatory element(s), adding strong promoters, inducible promoters or multiple promoters or removing regulatory sequences such that expression of the gene products is modified, modifying the chromosomal location of the operon, altering nucleic acid sequences adjacent to the operon or within the operon such as a ribosome binding site, increasing the copy number of the operon, modifying proteins (e.g., regulatory proteins, suppressors, enhancers, transcriptional activators and the like) involved in transcription of the operon and/or translation of the gene products of the operon, or any other conventional means of deregulating expression of genes routine in the art (including but not limited to use of antisense nucleic acid molecules, for example, to block expression of repressor proteins). Deregulation can also involve altering the coding region of one or more genes to yield, for example, an enzyme that is feedback resistant or has a higher or lower specific activity.

In another preferred embodiment, a recombinant organism, e.g., microorganism is designed or engineered such that at least one B6 vitamer biosynthetic enzyme, is overexpressed. The term “overexpressed” or “overexpression” includes expression of a gene product (e.g., a biosynthetic enzyme) at a level greater than that expressed prior to manipulation of the microorganism or in a comparable organism, e.g., microorganism which has not been manipulated. In one embodiment, the organism, e.g., the microorganism can be genetically designed or engineered to overexpress a level of gene product greater than that expressed in a comparable organism, e.g., microorganism which has not been engineered.

Genetic engineering can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins). Genetic engineering can also include deletion of a gene, for example, to block a pathway or to remove a repressor. In embodiments featuring organisms having deleted genes, the skilled artisan will appreciate that at least low levels of certain compounds may be required to be present in or added to the culture medium in order that the viability of the organism is not compromised. Often, such low levels are present in complex culture media as routinely formulated. Moreover, in processes featuring culturing organisms having deleted genes cultured under conditions such that commercially or industrially attractive quantities of product are produced, it may be necessary to supplement culture media with slightly increased levels of certain compounds.

In another embodiment, the microorganism can be physically or environmentally manipulated to overexpress a level of gene product greater than that expressed prior to manipulation of the organism or in a comparable microorganism which has not been manipulated. For example, a organism can be treated with or cultured in the presence of an agent known or suspected to increase transcription of a particular gene and/or translation of a particular gene product such that transcription and/or translation are enhanced or increased. Alternatively, a organism can be cultured at a temperature selected to increase transcription of a particular gene and/or translation of a particular gene product such that transcription and/or translation are enhanced or increased.

IV Culturing and Fermenting Recombinant Microorganisms

The term “culturing” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured in solid media or semi-solid media. In a preferred embodiment, a microorganism of the invention is cultured in media (e.g., a sterile, liquid media) comprising nutrients essential or beneficial to the maintenance and/or growth of the microorganism (e.g., carbon sources or carbon substrate, for example carbohydrate, hydrocarbons, oils, fats, fatty acids, organic acids, and alcohols; nitrogen sources, for example, peptone, yeast extracts, meat extracts, malt extracts, soy flour, urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, molybdenum, and/or cobalt salts; as well as growth factors such as amino acids, vitamins, and the like).

Preferably, microorganisms of the present invention are cultured under controlled pH. The term “controlled pH” includes any pH which results in production of the desired product. In one embodiment microorganisms are cultured at a pH of about 7. In another embodiment, microorganisms are cultured at a pH of between 6.0 and 8.5. The desired pH may be maintained by any number of methods known to those skilled in the art.

Also preferably, microorganisms of the present invention are cultured under controlled aeration. The term “controlled aeration” includes sufficient aeration (e.g., oxygen) to result in production of the desired product. In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the culture vessel (e.g., tube or flask) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents).

Moreover, microorganisms of the present invention can be cultured under controlled temperatures. The term “controlled temperature” includes any temperature which results in production of the desired product (e.g., a B6 vitamer). In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous processes or methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation media is added continuously to a fermentor and an equal amount of used or “conditioned” media is simultaneously removed, preferably for recovery of the desired product (e.g., a B6 vitamer). A variety of such processes have been developed and are well-known in the art.

The phrase “culturing under conditions such that a desired compound is produced” includes maintaining and/or growing microorganisms under conditions (e.g., temperature, pressure, pH, duration, etc.) appropriate or sufficient to obtain production of the desired compound or to obtain desired yields of the particular compound being produced. For example, culturing is continued for a time sufficient to produce the desired amount of a compound (e.g., a B6 vitamer). Preferably, culturing is continued for a time sufficient to substantially reach suitable production of the compound (e.g. a time sufficient to reach a suitable concentration of a B6 vitamer). In one embodiment, culturing is continued for about 12 to 24 hours. In another embodiment, culturing is continued for about 24 to 36 hours, 36 to 48 hours, 48 to 72 hours, 72 to 96 hours, 96 to 120 hours, 120 to 144 hours, or greater than 144 hours. The methodology of the present invention can further include a step of recovering a desired compound (e.g., a B6 vitamer). The term “recovering” a desired compound includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), dialysis, filtration, concentration, crystallization, recrystallization, pH adjustment, lyophilization and the like. For example, a compound can be recovered from culture media by first removing the microorganisms from the culture. The resulting solutions are then passed through or over a cation exchange resin to remove cations and/or through or over an anion exchange resin to purify or concentrate the desired product. The resulting compound can subsequently be converted to a salt (e.g., a chloride or sulfate salt) by ion exchange.

Preferably, a desired compound of the present invention is “extracted,” “isolated” or “purified” such that the resulting preparation is substantially free of other media components (e.g., free of media components and/or fermentation byproducts). The language “substantially free of other media components” includes preparations of the desired compound in which the compound is separated from media components or fermentation byproducts of the culture from which it is produced. In one embodiment, the preparation has greater than about 80% (by dry weight) of the desired compound (e.g., less than about 20% of other media components or fermentation byproducts), more preferably greater than about 90% of the desired compound (e.g., less than about 10% of other media components or fermentation byproducts), still more preferably greater than about 95% of the desired compound (e.g., less than about 5% of other media components or fermentation byproducts), and most preferably greater than about 98-99% desired compound (e.g., less than about 1-2% other media components or fermentation byproducts). When the desired compound has been derivatized to a salt, the compound is preferably further free of chemical contaminants associated with the formation of the salt. When the desired compound has been derivatized to an alcohol, the compound is preferably further free of chemical contaminants associated with the formation of the alcohol.

In an alternative embodiment, the desired compound is not purified from the microorganism, for example, when the microorganism is biologically non-hazardous (e.g., safe). For example, the entire culture (or culture supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

Preferably, a production method of the present invention results in production of the desired compound, e.g., a B6 vitamer, at a significantly high yield. The phrase “significantly high yield” includes a level of production or yield which is sufficiently elevated or above what is usual for comparable production methods, for example, which is elevated to a level sufficient for commercial production of the desired product (e.g., production of the product at a commercially feasible cost). In one embodiment, the invention features a production method that includes culturing a recombinant microorganism under conditions such that the desired product (e.g., a B6 vitamer) is produced at a level greater than 5 mg/L. In another embodiment, the invention features a production method that includes culturing a recombinant microorganism under conditions such that the desired product (e.g. a B6 vitamer) is produced at a level greater than 10 mg/L. In another embodiment, the invention features a production method that includes culturing a recombinant microorganism under conditions such that the desired product (e.g., a B6 vitamer) is produced at a level greater than 50 mg/L. In yet another embodiment, the invention features a production method that includes culturing a recombinant microorganism under conditions such that the desired product (e.g., a B6 vitamer) is produced at a level greater than 150 mg/L.

Depending on the biosynthetic enzyme or combination of biosynthetic enzymes manipulated, it may be desirable or necessary to provide (e.g., feed) microorganisms of the present invention at least one biosynthetic precursor such that the desired compound or compounds are produced. The term “biosynthetic precursor” or “precursor” includes an agent or compound which, when provided to, brought into contact with, or included in the culture medium of a microorganism, serves to enhance or increase biosynthesis of the desired product. In one embodiment, the biosynthetic precursor or precursor is glutamine. In another embodiment, the biosynthetic precursor or precursor is ribose. The amount of glutamine or ribose added is preferably an amount that results in a concentration in the culture medium sufficient to enhance productivity of the microorganism (e.g., a concentration sufficient to enhance production of a B6 vitamer). The term “excess ribose or glutamine” includes ribose or glutamine levels increased or higher that those routinely utilized for culturing the microorganism in question. For example, culturing the Bacillus microorganisms described in the instant Examples can be done in the presence of about 0-5 g/L ribose or glutamine. Accordingly, excess ribose or glutamine levels can include levels of about 5-10 g/L or more preferably about 5-20 g/L ribose or glutamine. Biosynthetic precursors of the present invention can be added in the form of a concentrated solution or suspension (e.g., in a suitable solvent such as water or buffer) or in the form of a solid (e.g. in the form of a powder). Moreover, biosynthetic precursors of the present invention can be added as a single aliquot, continuously or intermittently over a given period of time.

Methods for culturing other recombinant organisms, e.g., the recombinant plant organisms of the present invention, are known to one of skill in the art. For example, conditions for culturing and maintaining recombinant plant cells are described in Choi J. W., et al. (2001) Adv Biochem Eng Biotechnol 72: 63-102; James E., Lee J. M. (2001) Adv Biochem EngBiotechnol 72: 127-56; Fischer R. and Emans N. (2000) Transgenic Res. 9(4-5): 279-99; Fischer R., et al. (2000) J Biol Regul Homeost Agents April-June; 14(2): 83-92; Kutchan T. M. (1996) Gene 1996 November 7; 179(1): 73-81; Creissen G., et al. (1996) Biochem Soc Trans. May; 24(2): 465-9; McKenna T. S., et al. (1996) J Biotechnol January 26; 44(1-3): 83-9; Ma J. K. and Hein M. B. (1995) Plant Physiol October; 109(2): 341-6; Taticek R. A., Lee C. W., Shuler M. L. (1994) Curr Opin Biotechnol February; 5(2): 165-74; Arathoon W. R., Birch J. R. (1986) Science June 13; 232(4756): 1390-5; Murray K. (1980) Philos Trans R Soc Lond B Biol Sci August 11; 290(1040): 369-86. Furthermore, methods for culturing and maintaining the recombinant yeast organisms of the invention are also known to one of skill in the art, as described in, for example Kjeldsen T. (2000) Appl Microbiol Biotechnol September; 54(3): 277-86; Fischer R, et al. (1999) Biotechnol Appl Biochem October; 30 (Pt 2): 117-20; Mendoza-Vega O., Sabatie J, Brown S. W. (1994) FEMS Microbiol Rev December; 15(4): 369-410; Moir D. T., Mao J. I. (1990) Bioprocess Technol 9: 67-94.

This invention is further illustrated by the following examples which should not be construed as limiting. The contents of all references, patents and published patent applications cited throughout this application are incorporated herein by reference.

### EXAMPLES

### Example 1

### Biological Assay for B6 Vitamers Using Saccharomyces uvarum

Quantitation of B6 vitamers in supernatants of cultures of micro-organisms or extracts of organisms that have been genetically modified to increase production of B6 vitamers is conveniently done using Saccharomyces uvarum (formerly and still often named S. carlsbergensis) strain ATCC 9080 as the indicator strain or test organism. The method is essentially that described in the Difco Manual (1984, Difco Laboratories, Detroit, Mich., 10th Edition, pp. 1104-1106), with the modification that 50 mg/liter of streptomycin sulfate is added to the liquid growth medium for the test organism. However, any other appropriate indicator organism may be used, together with a medium that is appropriate for that organism that is free of B6 vitamers. For example, an E. coli pdxB mutant can be used in a standard minimal medium that is well known in the art, such as M9 glucose minimal medium (Miller, J., (1972) Experiments in Molecular Genetics, Cold Spring Harbor Laboratory Press, Cold Spring Harbor, N.Y.).

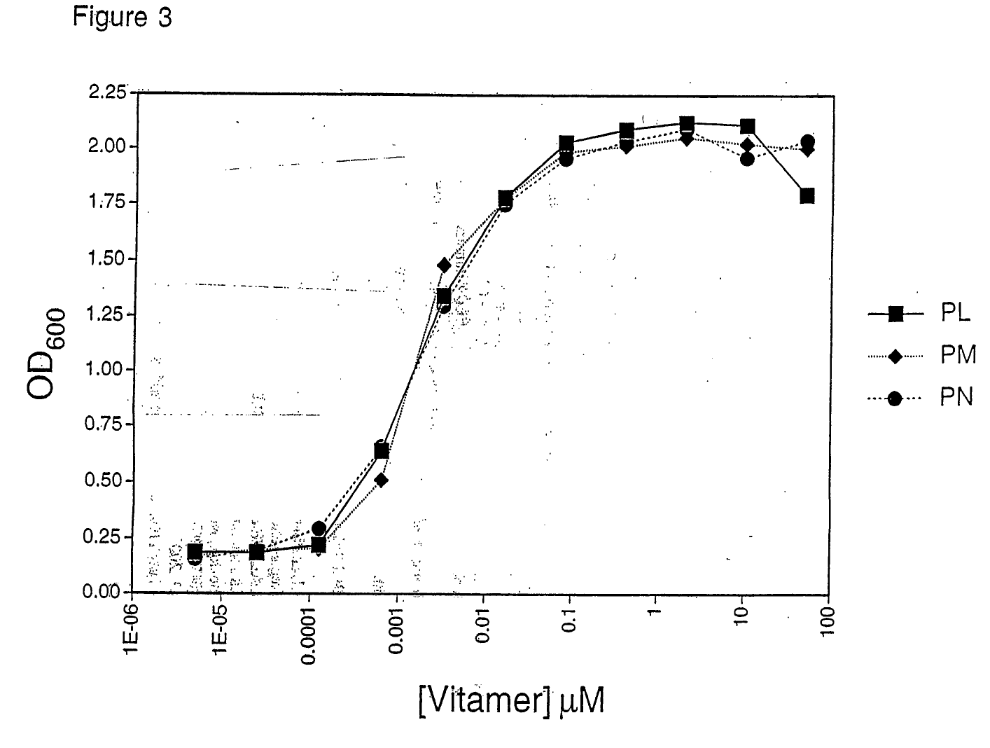

When using S. uvarum strain ATCC 9080 as the indicator strain, Bacto Pyridoxine Y Medium (Difco Laboratories, available through VWR Scientific, Inc.), supplemented with 50 mg/liter streptomycin sulfate, is used for the serial dilutions, and PN, PL, or PM is used to generate the standard curve. The responses to these three standard compounds are almost identical to each other with S. uvarum strain ATCC 9080 (FIG. 3).

### Example 2

### Deletion of a Portion of the yaaDE Operon in B. subtilis.

The SOR and SNO genes of Cercospora nicotianae were originally identified by mutations that lead to hypersensitivity to singlet oxygen-generating reagents (Ehrenschaft, M., et al. (1999) Proc. Natl. Acad. Sci. USA 96: 9347-9378). Mutations in either of these genes also lead to PL auxotrophy. The protein sequences obtained from translation of the SOR and SNO open reading frames were used as homology probes to search through the B. subtilis genome sequence using the BLAST homology search program of the Subtilist website. The SOR protein was significantly homologous to the YaaD protein, and the SNO protein was significantly homologous to the YaaE protein. Moreover, the genes encoding the YaaD and YaaE proteins (namely yaaD and yaaE) occur adjacent to each other on the B. subtilis chromosome as a two gene operon.

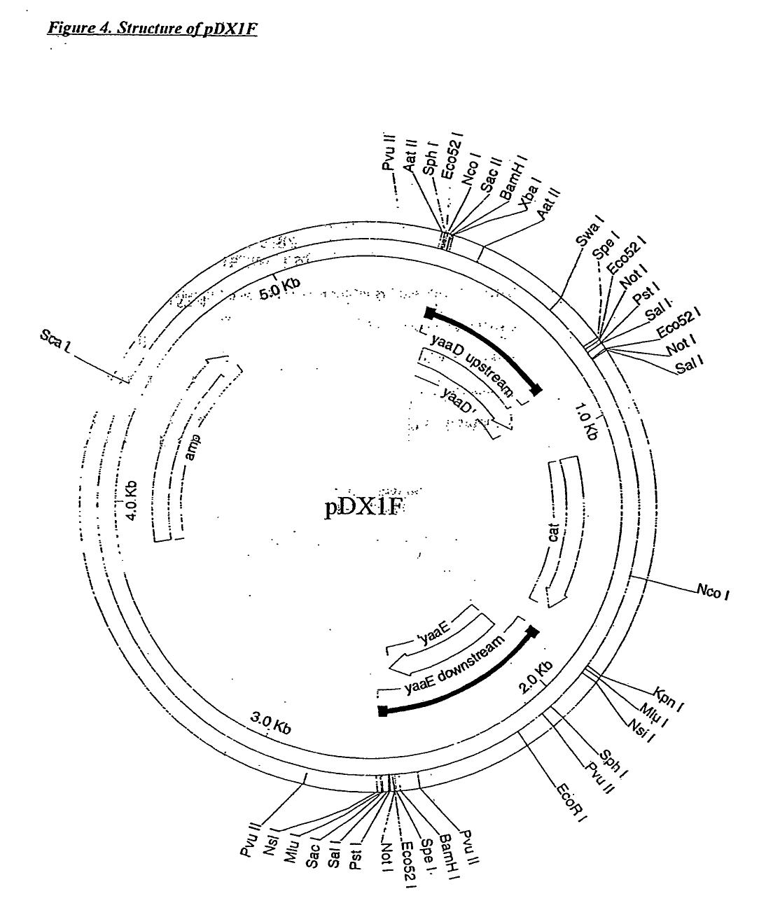

General methods for growth, storage, transformation, and molecular biology of B. subtilis strains are given in Harwood, C., and Cutting, S. (1990), Molecular Biological Methods for Bacillus, John Wiley and Sons, New York, N.Y., hereby incorporated in its entirety by reference. The yaaDE operon DNA sequence was amplified using the Polymerase Chain Reaction (PCR) with Pfu Turbo DNA polymerase (Stratagene, Inc., used according to the manufacturer's instructions). The DNA primers used were RY395 (SEQ ID NO:1) and RY396 (SEQ ID NO:2). RY395, the upstream primer, introduces an XbaI site and artificial ribosome binding site. RY396, the downstream primer, introduces a BamHI site. The template DNA was chromosomal DNA isolated form wild type B. subtilis strain PY79. The blunt ended PCR product was ligated into the EcoRV site of pGEM5Zf(+) (Promega, Inc.) to give plasmid pAN368. Next, a gram positive chloramphenicol resistance gene on a blunt DNA fragment was ligated into pAN368 that had been cut with HpaI, to give plasmid pDX1F (SEQ ID NO:5, FIG. 4). pDX1F therefore is deleted for a portion of yaaD and a portion of yaaE. pDX1F was used to transform wild type B. subtilis strain PY79 to 5 mg/liter chloramphenicol resistance, and a double crossover event was confirmed using PCR and the same primers used to clone yaaDE. The resulting strain was named PX1.

PX1 was able to grow on Spizizen's minimal medium with trace elements (SMM) (Harwood, C., and Cutting, S. (1990) Molecular Biological Methods for Bacillus, John Wiley and Sons, New York, N.Y., pp. 548-549) supplemented with 2 μM pyridoxal HCl (Sigma-Aldrich Chemical Co.), but it did not grow without the supplement. Thus it was established that at least one of yaaD or yaaE is required for PLP synthesis in B. subtilis.

### Example 3

### Deletion of yhaF in B. subtilis.

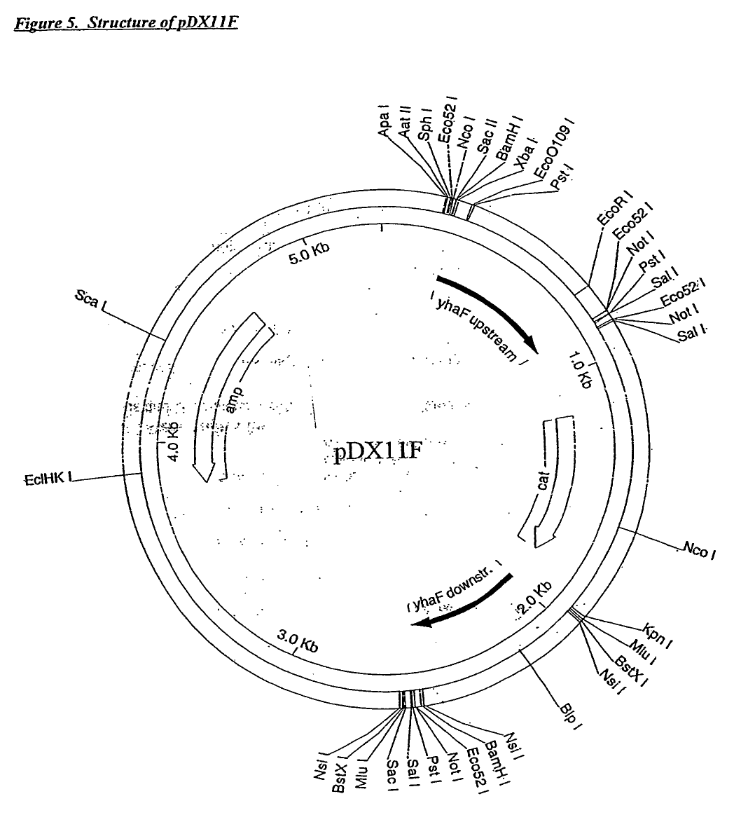

The protein sequence of the E. coli pdxF gene was used as a probe to search the B. subtilis genome as described in Example 1. The only significant homolog was yhaF. In a fashion similar to that of Example 1, the yhaF was cloned and deleted from the chromosome of PY79 using plasmid pDX11F (SEQ ID NO:6, FIG. 5), to give strain PX11. The PCR primers used to clone yhaF were RY407 (SEQ ID NO:3) and RY408 (SEQ ID NO:4). The restriction sites used for insertion of the antibiotic resistance gene were the PshA1 and EheI sites in the yhaF coding region. PX11 is a serine auxotroph, but not a PL auxotroph. By comparison to E. coli, it appears that yhaF functions in serine synthesis and probably encodes the equivalent of SerC, but that the YhaF protein is not required for PLP synthesis in B. subtilis. Therefore, it is established that sequence homology alone does not necessarily imply functional homology.

### Example 4

### Overexpression of the yaaDE Operon in B. subtilis.

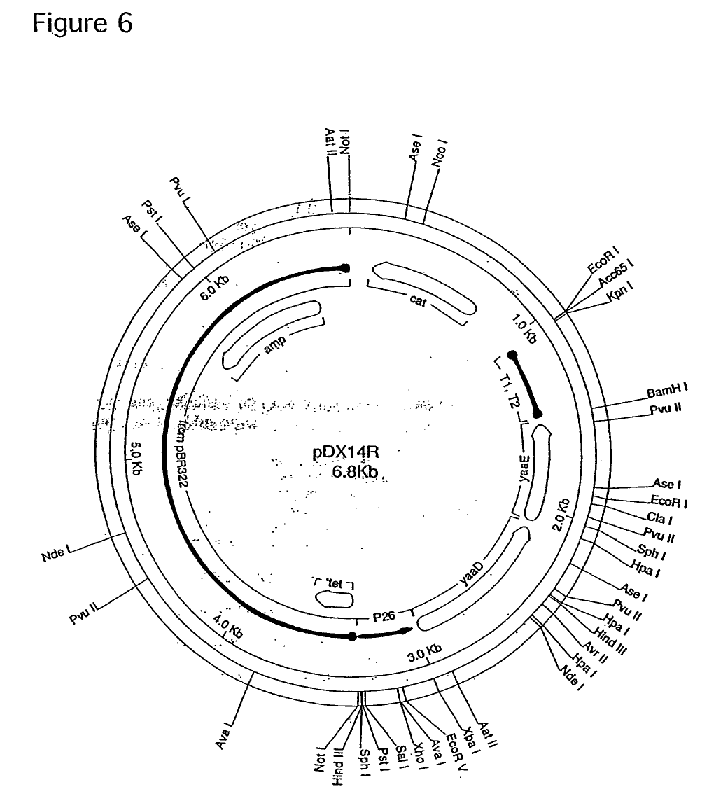

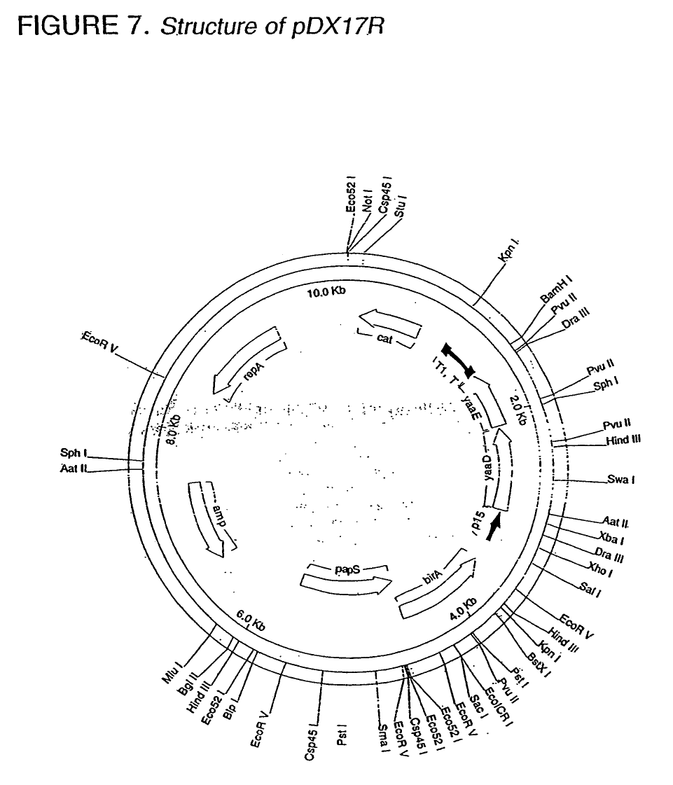

The XbaI to BamHI fragment from pAN368 that contains the yaaDE operon and artificial ribosome binding site was inserted into either of two expression vectors, to yield plasmids pDX14R (SEQ ID NO:7) and pDX17R (SEQ ID NO:8), respectively. In pDX14R and pDX17R, the yaaDE operon is expressed from the strong constitutive B. subtilis phage SP01 promoters, P₂₆ and P₁₅, respectively (see FIGS. 6 and 7).

pDX14R and pDX17R were each transformed into wild type B. subtilis strain PY79, selecting for chloramphenicol resistance. The plasmids integrate into the chromosome at the yaaDE locus by single crossover. The resulting strains were named PX14 and PX17, respectively.

PX14 and PX17 were grown for 48 hours at 37° C. in 5 ml test tube cultures in a roller drum at about 100 rotations per minute. The culture medium was SVY (20 g Difco Veal Infusion Broth, 5 g Difco Yeast Extract, 2 g ammonium sulfate, 5 g sodium glutamate, and 30 g glucose per liter, buffered with 200 mM potassium phosphate, pH 7.0). Cells were removed by centrifugation followed by sterile filtration (Millipore 0.45 micron), and the supernatant solutions were assayed for PL equivalents using the biological assay described in Example 1. The parent strain, PY79 was grown and processed in similar fashion as a control. The uncultured SVY medium was assayed as another control, since it was likely that the SVY medium contained a measurable level of B6 vitamers. The results are shown in Table 2, below.

TABLE 2
Production of B6 vitamers by Bacillus subtilits derivatives in 48
hour test tube cultures grown in SVY

| | | | | Total B6 | Net B6 |
|---|---|---|---|---|---|
| | | Integration | | Vitamers¹ | Vitamers² |
| Strain | Cassette | Target | OD₆₀₀ | mg/liter | mg/liter |
| PX14 | P₂₆yaaDE | yaaDE | 17 | 7.2 | 7.0 |
| PX17 | P₁₅yaaDE | yaaDE | 17 | 4.9 | 4.7 |
| PX1 | ΔyaaDE | yaaDE | 8 | 0.4 | 0.2 |
| PY79 | — | — | 19 | 0.8 | 0.6 |
| (Medium) | — | — | 0.08 | 0.2 | (0) |

¹Sum of PN, PL, PM, and derivatives thereof that can be utilized by pyridoxine indicator strain S. carlbergensis as a source of vitamin B6 for growth.
²Calculated by subtracting the amount assayed in the medium.

After subtracting the B6 vitamers contained in the medium, strain PX14 produced 7.0 mg/liter PL equivalents, while the parent PY79 produced only 0.6 mg/liter of PL equivalents. Thus, expression of the yaaDE operon has been shown to be rate limiting for B6 vitamer production in B. subtilis. Moreover, a genetically modified strain, where this rate limiting step was enhanced, produced more than a ten-fold increase in B6 vitamer secretion compared to that of the parent.

### Example 5

### Shake Flask Experiments to Determine B₆ Vitamer Production from B. subtilis Strain PX14 Containing an Amplifiable P₂₆ yaaDE Cassette

To further test B₆ production, PX14 and PY79 were grown in parallel in shake flasks in soy flour, maltose, MOPS medium (SMM). The 200 ml baffled shake flasks contained 40 ml of medium, were covered with 4× Bioshield covers, and shaken at 37° C. and 300 rpm. Samples were taken at 24 and 48 hours and analyzed for vitamer concentration by HPLC (Table 3).

Titers produced by the parental control strain PY79 were 3 mg/l. PX14 produced a B₆ titer of 45 mg/L when grown in shake flasks containing SMM. In shake flasks, where the overall vitamer concentration increased with time, the amount of PN produced increased and exceeded that of PMP at 48 hours (Table 3).

B. subtilis has a fairly broad temperature range of growth up to approximately 50° C. A significant increase of B₆ titer was seen with B. subtilis strain PX14 when grown in shake flasks with SMM at 43° C. as compared to 37° C. (Table 4). The B₆ titer increased more than 25% at 43° C. as compared to that at 37° C.

TABLE 3
Vitamer composition of PX14 and PY79 grown in shake flasks in
SMM³.

| Strain | Time point (hr.) | PMP¹ | PM¹ | PL¹ | PN¹ | Total Vitamer² |
|---|---|---|---|---|---|---|
| PX14 | 24 | 10.8 | 2.7 | 0.0 | 6.4 | 20 |
| | 48 | 15.5 | 8.5 | 1.7 | 19.0 | 45 |
| PY79 | 24 | 1.0 | 0.0 | 0.0 | 0.0 | 1 |
| | 48 | 2.9 | 0.4 | 0.0 | 0.0 | 3 |

¹PMP, PM, PL and PM are reported in mg/l and quantified by HPLC.
²Total vitamer is equal to the sum of PMP, PM, PL and PM and reported in mg/l.
³SMM contains in a final volume of 200 ml: 4.0 g soy flour, 1.6 g (NH₄)₂SO_{4,} 1.0 g monosodium glutamate, 2.0 ml 100 X PSTE trace minerals, 5.0 ml 4 M potassium phosphate buffer pH 7.2, 40 ml 1.5 M MOPS buffer pH 7.2, and deionized water.
Added after autoclaving at 20X concentrate to give a final concentration of 3% maltose with 5 mM magnesium chloride and 0.7 mM calcium chloride. 100 X PSTE contains 0.2 g/l MnCl₂.H₂0, .15 g/l ZnSO₄.7H₂0, 0.2 g/l CoCl₂.6H₂0, 0.025 CuSO₄.5H₂0 and 0.075 g/l Na₂MoO₄.2H₂0.

TABLE 4
Vitamer composition of PX14 grown in shake flasks in SMM for 48
hours.

| Strain | Temperature (° C.) | PMP¹ | PM¹ | PL¹ | PN¹ | Total Vitamer² |
|---|---|---|---|---|---|---|
| PX14 | 37 | 15 | 9 | 2 | 19 | 45 |
| | 43 | 11 | 13 | 1 | 36 | 61 |

¹PMP, PM, PL and PM are reported in mg/L and quantified by HPLC.
²Total vitamer is equal to the sum of PMP, PM, PL and PM and reported in mg/L.

### Example 6

### Complementation of E. coli pdx Mutants by Plasmids that Express the B. subtilis yaaDE Operon.

Plasmid pDX14R, described above in Example 4, was used to transform various E. coli strains that contained mutations that lacked function in each of the known genes involved in PLP biosynthesis (except for dxs, which is an essential gene for E. coli). The selection was for resistance to 250 mg/liter ampicillin. Each transformant was then tested for growth on minimal medium (SMM with 0.5% glucose, see Example 2) supplemented with 100 mg/liter serine, and compared to growth of its respective untransformed parent on the same medium. All mutations tested were complemented by pDX14R. Specifically, pdxA, pdxB, pdxF, pdxJ, and pdxH, were all complemented by pDX14R. Therefore, expression of the B. subtilis yaaDE operon in E. coli is sufficient for PLP biosynthesis, even in the absence of any one of the above functional pdx genes. Several important and unexpected conclusions or inferences can be drawn form these results. First, the substrate(s) for the enzyme(s) encoded by yaaDE must be present in E. coli, even when a biosynthetic intermediate normally used to make PLP is absent or greatly reduced. Second, PNP or PLP is possibly the product of the enzyme(s) encoded by yaaDE. Third, since an early block in the E. coli PLP biosynthetic pathway (for example that in a pdxB mutant) does not prevent yaaDE from complementation, the substrates for the enzyme(s) encoded by yaaDE are not likely to be the same as for the last step in PMP or PLP synthesis in wild type E. coli. These unexpected results lead to the possibility of producing B6 vitamers using B. subtilis yaaDE or the homologous genes from another organism (for example, but not limited to, SOR and SNO from Cercospora nicotianae or PDX1 and PDX2 from S. cerevisiae) in a heterologous host species, including, but not limited to, E. coli and Oryza sativa.

### Example 7

### Overexpression of the yaaDE Operon in E. coli.

Plasmids pDX14R and pDX17R were transformed into E. coli strain DH5α (New England Biolabs), selecting for ampicillin resistance. The transformants were grown for 48 hours in 5 ml test tube cultures at 37° C., and the supernatants were worked up as in Example 3. The assay results for PL equivalents are shown in Table 5, below.

TABLE 5
Production of B6 vitamers by Escherichia coli harboring plasmids
containing engineered Bacillus subtilits genes¹

| | | | Total B6 | Net B6 |
|---|---|---|---|---|
| | Plasmid | | Vitamers² | Vitamers³ |
| Strain | Cassette | OD₆₀₀ | mg/liter | mg/liter |
| DH5α | P₂₆yaaDE | 7.6 | 3.2 | 3.1 |
| DH5α | P₁₅yaaDE | 8.2 | 3.2 | 3.1 |
| DH5α | — | 9 | 0.1 | (0) |

¹E. coli test tube cultures are grown in SVY for 48 hours.
²Sum of PN, PL, PM, and derivatives thereof that can be utilized by pyridoxine indicator strain S. carlbergensis as a source of vitamin B6 for growth.
³Calculated by subtracting the amount assayed in DH5α not containing plasmid.

Thus it has been shown that the yaaD and yaaE genes can be expressed in a heterologous host strain, and B6 vitamers can still be overproduced. By extension of this approach, the yaaD and yaaE genes of B. subtilis can be overexpressed in any organism where an overexpression system exists, and in the resulting strains, B6 vitamers will be overproduced. Overproduction of the rate limiting enzyme for B6 vitamer production in any organism that is capable of producing B6 vitamers will lead to overproduction of B6 vitamers.

### Example 8

### Test Tube Experiment to Determine B₆ Vitamer Production from E. coli Strain DH5α (pDX14) Grown on SVY Supplemented with Pentose or Hexose Carbon Sources

In order to test whether feeding pentoses could enhance B₆ synthesis, E. coli strain DH5α harboring a plasmid containing the P₂₆ yaaDE cassette were grown in a variety of SVY based media containing a pentose (xylose or ribose) or hexose (glucose or maltose) as the carbohydrate source. The strains were also grown on SVY medium containing no carbohydrate source as a control. Production of B₆ vitamers from E. coli strain DH5α(pDX14) grown in SVY ribose is greater than that from E. coli strain DH5α(pDX14) grown in SVY xylose, glucose, arabinose or maltose. More than 60 mg/l of total B₆ vitamers have been produced in the DH5α(pDX14) cultures grown in SVY ribose test tube with PN as the major product (Table 6).

TABLE 6
Production of B₆ vitamers by E. coli harboring a plasmid containing
engineered B. subtilis genes and grown in the presence or absence of
hexose or pentose carbon sources.

| Strain | Sugar | OD₆₀₀ | Total Vitamer¹ |
|---|---|---|---|
| DH5α(pDX14) | Glucose | 9 | 8 |
| | Ribose | 16 | 61 |
| | Xylose | 8 | 31 |
| | Arabinose | 7 | 21 |
| | None | 7 | 25 |
| DH5α | Glucose | 9 | 4 |
| | Ribose | 18 | 5 |
| | Xylose | 9 | 4 |
| | Arabinose | 9 | 1 |
| | None | 7 | 3 |

¹Total vitamer concentration (mg/l) is quantified by HPLC and equal to the sum of PMP, PM, PL, and PN.

### Example 9

### Vitamin B₆ Titers of a pdxB E. coli Strain Producing YaaD in the Absence of YaaE

In order to determine the titers of B₆ produced from YaaD alone, in the absence of YaaE, E. coli strain WG1012 (pdxB) was transformed with pDX14 and pDX24 (plasmid isolates N2, D2 and D6), and control plasmid pBR322. These transformed strains were grown in 5 mL test tube cultures in SVY ribose in roller drums at 37° C. for 48 hours. Ribose was chosen as the carbon source, since past experiments showed that ribose improved B₆ vitamer production in E. coli transformants. B₆ titers of culture supernatants were determined by HPLC assay. In all cases, the plasmids that complemented the pdxB strain led to measurable production of B₆, whereas as the plasmids (i.e. pBR322) that did not complement did not lead to any measurable B₆ (Tables 7 and 8) Several isolates of pDX24, which contains the yaaD ORF, produced approximately 7-10 mg/L of the B₆ vitamers. However, E. coli strain WG1012 (pdxB) containing pDX24 produced only a fraction of the amount of B₆ as compared to the E. coli strain WG1012 harboring pDX14 containing the P₂₆ yaaDE cassette (Table 8).

Apparently, YaaD is functional by itself and can catalyze the synthesis of vitamers similar in composition to those synthesized by YaaDE, albeit less efficiently. It appears that while YaaE is not absolutely essential for B₆ synthesis, it provides a supportive role to YaaD, thereby leading to higher B₆ titers. YaaD is also able to function independently of YaaE in B. subtilis, however, less efficiently than when YaaE is present. Expressing a copy of P₂₆yaaD in either PX1 or PY79 resulted in higher titers of B₆ vitamers as compared to the respective parental controls.

TABLE 7
YaaD complements a pdxB E. coli auxotroph.

| Strain and | | | PIX free + |
|---|---|---|---|
| plasmid | Cassette | PIX free | 2 μM PL |
| E. coli pdxB⁻ | | | |
| pBR322 | None | − | + |
| pDX14 | P₂₆yaaDE | + | + |
| pDX24 | P₂₆yaaD | + | + |

TABLE 8
Vitamer profile of E. coli pdxB⁻ strains harboring a plasmid
containing a P₂₆yaaDE or a P₂₆yaaD cassette.

| | Iso- | | | | | | PM + PL + PN |
|---|---|---|---|---|---|---|---|
| Plasmid | late | Cassette | OD₆₀₀ | PM | PL | PN | (mg/L) |
| pDX14 | | P₂₆yaaDE | 14.1 | 0.6 | 0.0 | 28.8 | 29.4 |
| pDX24 | N2 | P₂₆yaaD | 12.2 | 0.0 | 2.0 | 7.4 | 9.4 |
| | D2 | P₂₆yaaD | 15.5 | 0.0 | 0.0 | 6.9 | 6.9 |
| | D6 | P₂₆yaaD | 13.6 | 0.0 | 0.0 | 9.7 | 9.7 |
| pBR322 | | — | 12.4 | 0.0 | 0.0 | 0.0 | 0.0 |
| SVY- | | | — | 0.0 | 0.0 | 0.0 | 0.0 |
| ribose | | | | | | | |

5 mL test tube cultures were grown in SVY-ribose and roller drums at approximately 100 RPM, 37° C. for 48 hours. Vitamers were quantified by HPLC.

The YaaD and YaaE protein sequences were used as probes to search the NCBI database for homologs in plants using the BLAST™ program which can be found at the National Center for Biotechnology Information website (Altschul S. F (1990) J. Mol. Biol. 215(3): 403-10). Several homologs of YaaD were found in several genera of plants, including Arabidopsis, Oryza, Ginkgo, Hevea, Phaseolus, and Stellaria. Two homologs of YaaE were found in Arabidopsis thaliana. However, no homologs of pdxA and pdxJ were found. Therefore, the plant kingdom appears to use the Type B Pathway for B6 vitamer biosynthesis. Thus for example, overexpression of the YaaD homolog (GenBank accession number AAL73561) from Oryza sativa (rice), and the A. thaliana homolog of YaaE (GenBank accession number AB011483) together in a plant using methods well know in the art, such as expression from the Cauliflower Mosaic Virus 35S promoter, will lead to overproduction of B6 vitamers in that plant.

### Example 10

### Other Routes to Increasing the Activity of Enzymes Involved in B6 Vitamer Synthesis

The overexpression of the yaaDE operon leads to an increase in the amount of the encoded enzyme(s), which in turn leads to an increase in the total activity of said enzyme(s). Increase in this activity leads to an increase in the production B6 vitamer. Other methods can be used to increase the activity of the relevant enzyme(s) under conditions of B6 vitamer production. For example, in addition to increasing the amount of a relevant enzyme(s), the total activity of the relevant enzyme(s) can be increased by mutating the gene(s) to increase the specific activity of the enzyme(s), and/or by mutating the gene(s) to encode a feedback resistant variant of the enzyme(s). Such desirable mutations can be obtained by screening large numbers of mutants for the increased activity as evidenced by an increase in B6 vitamer production as described in Example 4, or by selecting for mutants that are resistant to inhibitors that are specific for the PLP biosynthetic pathway, and screening among those mutants for an increase in B6 vitamer production. Examples of such inhibitors are isoniazid, iproniazid, and ginkgotoxin (4′-methoxy pyridoxine) (Dempsey and Arcement (1971) J. Bacteriol. 107(2): 580-582; Pflug, W., and Lingens, F., (1978) Hoppe-Seyler's Z. Physiol. Chem. 359: 559-570; Fiehe, K., et al., (2000) J. Nat. Prod. 63(2): 185-189).

### Example 11

### Processing of Biosynthetic B6 Vitamers

A B6 vitamer produced by a genetically modified organism of the invention can be harvested and processed into a format that is appropriate for commercial use. For example, after culturing a B6 vitamer producing micro-organism in liquid culture, the entire culture, including cells can be dried by evaporation or by spray drying, and the resulting powder can be mixed into animal feeds. Alternatively, the cells can be first removed by centrifugation or filtration, and the resulting supernatant solution can be dried as described above. As another alternative, the B6 vitamer can be purified from the culture broth by techniques well known in the art, such as filtration, reverse osmosis, column chromatography (ion exchange, hydrophobic or hyrophilic adsorption, gel filtration, etc.), solvent extraction, precipitation, distillation, evaporation, and the like. If the B6 vitamer producing organism is a plant, then the appropriate portion of the plant (for example the leaves, stems, roots, flowers, fruits, seeds, or any combination thereof) can be harvested and processed. For example the plant material can be dried and used directly, or the material can be pulverized or ground and the B6 vitamer extracted and/or processed as described above for cultures.

The production organism can be a micro-organism that normally inhabits the gut of humans or an animal if interest (for example one of many bacteria of the genus Lactobacillus, such as L. acidophilus), and the B6 vitamer can be delivered by ingestion of the organism.

### Example 12

### Genes Homologous to yaaDE Expressed in Host Organisms for the Production of B6

Preferred methods for the production of vitamin B6 involve expression of Bacillus subtilis yaaD and/or yaaE or expression of genes that are homologous to Bacillus subtilis yaaD and/or yaaE in host organisms (type A or type B organisms) such as E. coli, S. cerevisiae, Arabidopsis thaliana, etc. In order to identify genes or proteins that are homologous to yaaD and yaaE a procedure such as a protein or nucleotide BLAS™ (Basic Alignment Search Tool) search can be implemented (Altschul, Stephen F., et al. (1997) Nucleic Acids Res. 25: 3389-3402). The BLAST™ search tool and can be found and used at the National Center for Biotechnology Information website. BLAST™ is an algorithm designed to compare sequences and provide sequence alignments between two or more previously characterized sequences and also provides a method for rapid searching of nucleotide and protein databases. A typical description line of a BLAST output table is given as “gi|586857|sp|P37527|YAAD_BACSU 31.5 kDa guanylylated protei . . . 506 e-142” where “gi” is the GenBank identifier of the subject which in this case is number “586857”, “sp” represents the databank from which the subject was retrieved, “P37527” represents the GenBank accession number and “YAAD_BACSU 31.5 kDa guanylylated protei . . . ” is a brief description of the sequence, the number “506” represents the bit score of the highest-scoring HSP's (high scoring pairs) and “e-142” is the E value that represents the statistical significance of a given alignment. The E value also reflects the size of the database and the scoring value used. The lower the E value, the more significant the homology. The description lines in the BLAST™ output table (Table 9, below) represents a typical BLAST™ query of the primary amino acid sequence of B. subtilis YaaD and is sorted by increasing E value with the most significant alignments (lowest E values) at the top. A similar BLAST™ query of primary amino acid sequence of B. subtilis YaaE is represented in Table 10, below. These alignments represent a small portion of the protein sequences homologous to YaaD and YaaE protein sequences from various organisms as the sequences from many organisms have not been, or only partially, categorized into the current databases.

TABLE 9
Sequences producing significant alignments to Bacillus subtilis YaaD

| | Score | E |
|---|---|---|
| | (bits) | Value |
| gi\|586857\|sp\|P37527\|YAAD_BACSU 31.5 kDa guanylylated protei . . . | 506 | e−142 |
| gi\|21397980\|ref\|NP_653965.1\| SOR_SNZ, SOR/SNZ family [Bacil . . . | 453 | e−126 |
| gi\|10172634\|dbj\|BAB03741.1\| superoxide-inducible protein [B . . . | 437 | e−122 |
| gi\|15603097\|ref\|NP_246169.1\| unknown [Pasteurella multocida . . . | 435 | e−121 |
| gi\|16414718\|emb\|CAC97434.1\| similar to a protein required f . . . | 431 | e−120 |
| gi\|22778374\|dbj\|BAC14643.1\| superoxide-inducible protein 7( . . . | 430 | e−119 |
| gi\|16411571\|emb\|CAD00179.1\| similar to a protein required f . . . | 428 | e−119 |
| gi\|25364956\|pir\|\|H89818 conserved hypothetical protein SA04 . . . | 424 | e−118 |
| gi\|22090614\|dbj\|BAC06852.1\| superoxide-inducible protein [B . . . | 418 | e−116 |
| gi\|23038401\|gb\|ZP_00070557.1\| hypothetical protein [Oenococ . . . | 392 | e−108 |
| gi\|7482301\|pir\|\|F69188 ethylene-inducible protein - Methano . . . | 360 | 1e−98 |
| gi\|22970419\|gb\|ZP_00017505.1\| hypothetical protein [Chlorof . . . | 359 | 3e−98 |
| gi\|6459121\|gb\|AAF10938.1\|AE001982_12 singlet oxygen resista . . . | 346 | 2e−94 |
| gi\|13310484\|gb\|AAK18310.1\|AF344827_1 Sor-like protein [Gink . . . | 342 | 5e−93 |
| gi\|23017679\|gb\|ZP_00057403.1\| hypothetical protein [Thermob . . . | 340 | 1e−92 |
| gi\|18481963\|gb\|AAL73561.1\|AC079632_5 Putative ethylene-indu . . . | 339 | 2e−92 |
| gi\|11358736\|pir\|\|T48163 pyridoxine biosynthesis protein-lik . . . | 338 | 4e−92 |
| gi\|3123099\|sp\|O14027\|YEM4_SCHPO Hypothetical protein C29B12 . . . | 338 | 7e−92 |
| gi\|11359355\|pir\|\|T46647 pyridoxine biosynthesis protein pyr . . . | 338 | 8e−92 |
| gi\|2129913\|pir\|\|S60047 ethylene-responsive protein 1 - Para . . . | 337 | 1e−91 |
| gi\|10719739\|gb\|AAG17942.1\| putative pyridoxine biosynthetic . . . | 337 | 2e−91 |
| gi\|11362638\|pir\|\|T46646 pyridoxine biosynthesis protein pdx . . . | 336 | 2e−91 |
| gi\|20094807\|ref\|NP_614654.1\| Pyridoxine biosynthesis enzyme . . . | 336 | 3e−91 |
| gi\|7462114\|pir\|\|A72372 conserved hypothetical protein - The . . . | 335 | 4e−91 |
| gi\|21617922\|gb\|AAM66972.1\| pyridoxine biosynthesis protein . . . | 335 | 5e−91 |
| gi\|2193896\|emb\|CAB09637.1\| hypothetical protein MLCL581.12c . . . | 333 | 1e−90 |
| gi\|20807305\|ref\|NP_622476.1\| Pyridoxine biosynthesis enzyme . . . | 333 | 2e−90 |
| gi\|15827141\|ref\|NP_301404.1\| putative pyridoxine biosynthes . . . | 332 | 3e−90 |
| gi\|13878067\|gb\|AAK44111.1\|AF370296_1 putative SOR1 from the . . . | 332 | 3e−90 |
| gi\|21220023\|ref\|NP_625802.1\| conserved hypothetical protein . . . | 330 | 1e−89 |
| gi\|18076239\|emb\|CAC81977.1\| err-related and stress induced . . . | 330 | 2e−89 |
| gi\|20090425\|ref\|NP_616500.1\| pyridoxine biosynthesis protei . . . | 328 | 4e−89 |
| gi\|6137073\|emb\|CAB59635.1\| ethylene responsive receptor, ER . . . | 328 | 5e−89 |
| gi\|23052101\|gb\|ZP_00078783.1\| hypothetical protein [Methano . . . | 327 | 9e−89 |
| gi\|3123042\|sp\|O06208\|YQ06_MYCTU Hypothetical protein Rv2606 . . . | 327 | 2e−88 |
| gi\|15842146\|ref\|NP_337183.1\| pyridoxine biosynthesis protei . . . | 327 | 2e−88 |
| gi\|25364954\|pir\|\|A96973 probable phosphate-utilizing enzyme . . . | 323 | 2e−87 |
| gi\|23335752\|gb\|ZP_00120985.1\| hypothetical protein [Bifidob . . . | 322 | 4e−87 |
| gi\|21323554\|dbj\|\|BAB98181.1\| Pyridoxine biosynthesis enzyme . . . | 322 | 5e−87 |
| gi\|19552014\|ref\|NP_600016.1\| pyridoxine biosynthesis enzyme . . . | 322 | 5e−87 |
| gi\|23465712\|ref\|NP_696315.1\| widely conserved protein in up . . . | 320 | 1e−86 |
| gi\|21228534\|ref\|NP_634456.1\| putative pyridoxine biosynthes . . . | 318 | 5e−86 |
| gi\|2501580\|sp\|Q58090\|Y677_METJA Hypothetical protein MJ0677 . . . | 317 | 1e−85 |
| gi\|23493620\|dbj\|BAC18589.1\| conserved hypothetical protein . . . | 314 | 1e−84 |
| gi\|13541829\|ref\|NP_111517.1\| Predicted phosphate-utilizing . . . | 313 | 2e−84 |
| gi\|18893665\|gb\|AAL81653.1\| ethylene-inducible protein homol . . . | 312 | 4e−84 |
| gi\|14591161\|ref\|NP_143237.1\| ethylene-responsive protein [P . . . | 311 | 6e−84 |
| gi\|14521000\|ref\|NP_126475.1\| ETHYLENE-RESPONSIVE PROTEIN [P . . . | 311 | 7e−84 |
| gi\|15622519\|dbj\|BAB66510.1\| 336aa long hypothetical stress- . . . | 308 | 4e−83 |
| gi\|6015903\|emb\|CAB57730.1\| hypothetical protein [Sulfolobus . . . | 307 | 1e−82 |
| gi\|10639689\|emb\|CAC11661.1\| probable pyridoxine biosynthesi . . . | 304 | 1e−81 |
| gi\|1078513\|pir\|\|S55082 hypothetical protein YMR096w - yeast . . . | 303 | 2e−81 |
| gi\|14972957\|gb\|AAK75562.1\| pyridoxine biosynthesis protein . . . | 302 | 5e−81 |
| gi\|7483295\|pir\|\|D69313 ethylene-inducible protein homolog - . . . | 301 | 7e−81 |
| gi\|15458966\|gb\|AAL00126.1\| Pyridoxine biosynthesis protein . . . | 301 | 9e−81 |
| gi\|19704795\|ref\|NP_604357.1\| pyridoxine biosynthesis protei . . . | 299 | 4e−80 |
| gi\|1074877\|pir\|\|F64173 hypothetical protein HI1647 - Haemop . . . | 298 | 5e−80 |
| gi\|12802367\|gb\|AAK07850.1\|AF309689_12 Snz-type pyridoxine V . . . | 297 | 1e−79 |
| gi\|3172534\|gb\|AAC18606.1\| hypothetical protein IP1 [Francis . . . | 294 | 1e−78 |
| gi\|22405887\|gb\|ZP_00000750.1\| hypothetical protein [Ferropl . . . | 293 | 2e−78 |
| gi\|1730655\|sp\|P53824\|SNZ2_YEAST SNZ2 PROTEIN >gi\|6323996\|re . . . | 293 | 2e−78 |
| gi\|1084717\|pir\|\|S56196 hypothetical protein YFL059w - yeast . . . | 292 | 3e−78 |
| gi\|25364947\|pir\|\|A84331 hypothetical protein Vng1793c [impo . . . | 288 | 9e−77 |
| gi\|23479541\|gb\|EAA16340.1\| ethylene-inducible protein hever . . . | 288 | 9e−77 |
| gi\|23612291\|ref\|NP_703871.1\| pyridoxine biosynthetic enzyme . . . | 287 | 1e−76 |
| gi\|14600562\|ref\|NP_147079.1\| ethylene-responsive protein 1 . . . | 285 | 5e−76 |
| gi\|18313608\|ref\|NP_560275.1\| stress induced protein, conjec . . . | 279 | 4e−74 |
| gi\|4103954\|gb\|AAD01898.1\| A37 [Arabidopsis thaliana] >gi\|41 . . . | 249 | 2e−65 |
| gi\|2501581\|sp\|Q50841\|YTR5_METVA Hypothetical protein in tRN . . . | 237 | 1e−61 |
| gi\|44737\|emb\|CAA25434.1\| unidentified reading frame [Methan . . . | 218 | 6e−56 |
| gi\|2501579\|sp\|Q41348\|H47_STELP HYPOTHETICAL PROTEIN H47 . . . | 207 | 1e−52 |
| gi\|7488891\|pir\|\|S71492 ethylene-responsive protein 2 - Para . . . | 205 | 5e−52 |
| gi\|297176\|emb\|CAA50602.1\| expressed ORF [Stellaria longipes . . . | 196 | 4e−49 |
| gi\|21671884\|gb\|AAM74246.1\|AC074355_8 Putative protein simil . . . | 100 | 2e−20 |
| gi\|20521394\|dbj\|BAB91905.1\| putative ethylene-responsive pro . . . | 90 | 4e−17 |
| gi\|22138453\|gb\|AAM93437.1\| putative ethylene-inducible prot . . . | 84 | 3e−15 |
| gi\|3335369\|gb\|AAC27170.1\| similar to SOR1 from the fungus C . . . | 78 | 1e−13 |
| gi\|15217264\|gb\|AAK92608.1\|AC078944_19 Hypothetical protein . . . | 57 | 2e−07 |

TABLE 10
Sequences producing significant alignments to Bacillus subtilis YaaE

| | Score | E |
|---|---|---|
| | (bits) | Value |
| gi\|467402\|dbj\|BAA05248.1\| unknown [Bacillus subtilis] >gi\|2 . . . | 382 | e−105 |
| gi\|21397981\|ref\|NP_653966.1\| SNO, SNO glutamine amidotransf . . . | 258 | 3e−68 |
| gi\|10172635\|dbj\|BAB03742.1\| amidotransferase [Bacillus halo . . . | 231 | 4e−60 |
| gi\|22090615\|dbj\|BAC06853.1\| 2-deoxy-scyllo-inosose synthase . . . | 221 | 4e−57 |
| gi\|23038400\|gb\|ZP_00070556.1\| hypothetical protein [Oenococ . . . | 216 | 1e−55 |
| gi\|14246288\|dbj\|BAB56682.1\| conserved hypothetical protein . . . | 206 | 1e−52 |
| gi\|16414719\|emb\|CAC97435.1\| lin2206 [Listeria innocual] >gi\| . . . | 190 | 1e−47 |
| gi\|15603098\|ref\|NP_246170.1\| unknown [Pasteurella multocida . . . | 190 | 1e−47 |
| gi\|22778373\|dbj\|BAC14642.1\| amidotransferase [Oceanobacillu . . . | 187 | 5e−47 |
| gi\|15643238\|ref\|NP_228282.1\| amidotransferase, putative [Th . . . | 187 | 6e−47 |
| gi\|16411572\|emb\|CAD00180.1\| lmo2102 [Listeria monocytogenes . . . | 184 | 6e−46 |
| gi\|21220022\|ref\|NP_625801.1\| conserved hypothetical protein . . . | 176 | 1e−43 |
| gi\|2145628\|pir\|\|S72721 amidotransferase hisH homolog - Myco . . . | 176 | 2e−43 |
| gi\|7447522\|pir\|\|C70570 hypothetical protein Rv2604c - Mycob . . . | 176 | 2e−43 |
| gi\|13092704\|emb\|CAC29982.1\| conserved hypothetical protein . . . | 176 | 2e−43 |
| gi\|18977900\|ref\|NP_579257.1\| imidazoleglycerol-phosphate sy . . . | 174 | 7e−43 |
| gi\|6459120\|gb\|AAF10937.1\|AE001982_11 amidotransferase HisH, . . . | 172 | 2e−42 |
| gi\|23017678\|gb\|ZP_00057402.1\| hypothetical protein [Thermob . . . | 172 | 3e−42 |
| gi\|7447524\|pir\|\|D71007 hypothetical protein PH1354 - Pyroco . . . | 162 | 2e−39 |
| gi\|9757756\|dbj\|BAB08237.1\| amidotransferase hisH-like prote . . . | 162 | 2e−39 |
| gi\|14521001\|ref\|NP_126476.1\| imidazoleglycerol-phosphate sy . . . | 160 | 6e−39 |
| gi\|21228535\|ref\|NP_634457.1\| Imidazoleglycerol-phosphate sy . . . | 160 | 7e−39 |
| gi\|23052100\|gb\|ZP_00078782.1\| hypothetical protein [Methano . . . | 160 | 8e−39 |
| gi\|25314121\|pir\|\|H95170 conserved hypothetical protein SP14 . . . | 157 | 8e−38 |
| gi\|19915437\|gb\|AAM04979.1\| pyridoxine biosynthesis protein . . . | 156 | 1e−37 |
| gi\|15458965\|gb\|AAL00125.1\| Conserved hypothetical protein [ . . . | 156 | 2e−37 |
| gi\|18424366\|ref\|NP_568922.1\| imidazoleglycerol-phosphate sy . . . | 155 | 2e−37 |
| gi\|23465711\|ref\|NP_696314.1\| conserved hypothetical protein . . . | 152 | 3e−36 |
| gi\|23335751\|gb\|ZP_00120984.1\| hypothetical protein [Bifidob . . . | 151 | 5e−36 |
| gi\|13540888\|ref\|NP_110576.1\| Predicted glutamine amidotrans . . . | 149 | 2e−35 |
| gi\|21323555\|dbj\|BAB98182.1\| Predicted glutamine amidotransf . . . | 145 | 4e−34 |
| gi\|2650108\|gb\|AAB90721.1\| imidazoleglycerol-phosphate synth . . . | 144 | 8e−34 |
| gi\|20515816\|gb\|AAM24079.1\| predicted glutamine amidotransfe . . . | 143 | 2e−33 |
| gi\|16081194\|ref\|NP_393487.1\| hypothetical protein [Thermopl . . . | 142 | 2e−33 |
| gi\|2128068\|pir\|\|C64507 hypothetical protein homolog MJ1661 . . . | 142 | 3e−33 |
| gi\|5103636\|dbj\|BAA79157.1\| 186aa long hypothetical protein . . . | 139 | 2e−32 |
| gi\|25314111\|pir\|\|C84409 imidazoleglycerol-phosphate synthas . . . | 139 | 3e−32 |
| gi\|15023464\|gb\|AAK78573.1\|AE007574_11 Glutamine amidotransp . . . | 139 | 3e−32 |
| gi\|20094498\|ref\|NP_614345.1\| predicted glutamine amidotrans . . . | 138 | 3e−32 |
| gi\|7447523\|pir\|\|F69120 conserved hypothetical protein MTH19 . . . | 132 | 3e−30 |
| gi\|1574496\|gb\|AAC23295.1\| conserved hypothetical protein [H . . . | 131 | 4e−30 |
| gi\|19114059\|ref\|NP_593147.1\| conserved hypothetical protein . . . | 127 | 7e−29 |
| gi\|12802368\|gb\|AAK07851.1\|AF309689_13 Sno-type pyridoxine v . . . | 124 | 7e−28 |
| gi\|9954418\|gb\|AAG09049.1\|AF294268_1 pyridoxine synthesis pr . . . | 124 | 9e−28 |
| gi\|18076241\|emb\|CAC81978.1\| SNO protein [Suberites domuncula] . . . | 124 | 1e−27 |
| gi\|18076243\|emb\|CAC81976.1\| SNO protein [Suberites domuncula] . . . | 122 | 2e−27 |
| gi\|25314108\|pir\|\|H90203 glutamine amidotransferase, probabl . . . | 120 | 9e−27 |
| gi\|22970420\|gb\|ZP_00017506.1\| hypothetical protein [Chlorof . . . | 120 | 1e−26 |
| gi\|22405886\|gb\|ZP_00000749.1\| hypothetical protein [Ferropl . . . | 117 | 8e−26 |
| gi\|13937025\|gb\|AAK50016.1\|AF363613_1 pyridoxine [Aspergillu . . . | 114 | 6e−25 |
| gi\|18313609\|ref\|NP_560276.1\| conserved hypothetical protein . . . | 112 | 3e−24 |
| gi\|15921733\|ref\|NP_377402.1\| 200aa long conserved hypotheti . . . | 111 | 6e−24 |
| gi\|1302459\|emb\|CAA96268.1\| ORF YNL334c [Saccharomyces cerev . . . | 103 | 1e−21 |
| gi\|1084718\|pir\|\|S56195 probable membrane protein YFL060c - . . . | 103 | 2e−21 |
| gi\|1078512\|pir\|\|S55081 hypothetical protein YMR095c - yeast . . . | 102 | 4e−21 |
| gi\|23112427\|gb\|ZP_00097910.1\| hypothetical protein [Desulfi . . . | 101 | 7e−21 |
| gi\|23489463\|gb\|EAA21585.1\| Glutamine amidotranspherase [Pla . . . | 85 | 6e−16 |
| gi\|21228132\|ref\|NP_634054.1\| Amidotransferase hisH [Methano . . . | 51 | 8e−06 |
| gi\|20089791\|ref\|NP_615866.1\| imidazoleglycerol-phosphate sy . . . | 50 | 1e−05 |
| gi\|23023420\|gb\|ZP_00062656.1\| hypothetical protein [Leucono . . . | 50 | 2e−05 |
| gi\|23025661\|gb\|ZP_00064612.1\| hypothetical protein [Leucono . . . | 50 | 2e−05 |
| gi\|22999839\|gb\|ZP_00043799.1\| hypothetical protein [Magneto . . . | 49 | 4e−05 |
| gi\|12229838\|sp\|Q9P4P9\|HIS5_EMENI Imidazole glycerol phospha . . . | 49 | 6e−05 |
| gi\|23129284\|gb\|ZP_00111116.1\| hypothetical protein [Nostoc . . . | 48 | 8e−05 |
| gi\|25287041\|pir\|\|AE1977 amidotransferase [imported] - Nosto . . . | 48 | 9e−05 |
| gi\|22776228\|dbj\|BAC12505.1\| amidotransferase [Oceanobacillu . . . | 47 | 1e−04 |
| gi\|22776424\|dbj\|BAC12700.1\| phosphoribosylformylglycinamidi . . . | 47 | 2e−04 |
| gi\|14916682\|sp\|Q9UXW5\|PURQ_PYRAB Phosphoribosylformylglycin . . . | 46 | 2e−04 |
| gi\|11258455\|pir\|\|H81274 amidotransferase Cj1315c [imported] . . . | 46 | 2e−04 |
| gi\|23109765\|gb\|ZP_00095937.1\| hypothetical protein [Novosph . . . | 46 | 3e−04 |
| gi\|12229843\|sp\|Q9RDX3\|HIS5_LEGPN Imidazole glycerol phospha . . . | 45 | 4e−04 |
| gi\|23112136\|gb\|ZP_00097663.1\| hypothetical protein [Desulfi . . . | 45 | 6e−04 |
| gi\|15559183\|gb\|AAK58487.1\| unknown [Campylobacter jejuni] . . . | 45 | 6e−04 |

### Example 13

### Characterizing Functional B. subtilis yaaD and yaaE Homologs by Complementation of E. coli pdx Mutants by Plasmids that Express the B. subtilis yaaD and/or yaaE Homologs

In order that any given B. subtilis yaaD and/or yaaE homolog has similar function as the B. subtilis genes, the method of rescuing E. coli or other pdx auxotrophs can be employed. Briefly, DNA homologous to yaaD and yaaE sequences or DNA encoding proteins homologous to YaaD and/or YaaE, identified by BLAST™ or other methods, can be amplified using the Polymerase Chain Reaction (PCR) with Pfu Turbo DNA polymerase (Stratagene Inc., used according to the manufacturers instructions) using appropriately designed DNA primers. In an analogous method to the overexpression of the yaaDE operon in B. subtilis described in Example 4, a DNA fragment that contains a homologous gene or operon (Type B pathway) and artificial ribosome binding site can be inserted into an appropriate expression vector for example either of two expression vectors from Example 4. In these plasmids the yaaDE homologs would be expressed from a strong constitutive B. subtilis phage SP01 promoter such as P₁₅ or P₂₆. The resulting plasmids can be used to transform various E. coli or B. subtilis strains that contain mutations that lack function in known genes involved in PLP synthesis. The transformants can be selected for resistance to an appropriate antibiotic (such as ampicillin) that is encoded by the plasmid harboring the yaaDE homologs. Transformants can be tested for growth on minimal medium (SMM with 0.5% glucose) supplemented with 100 mg/L serine, and compared to growth of its respective untransformed parent on the same medium. True transformants (as opposed to revertants) that grow on minimal medium without any added B6 vitamer(s), can be concluded to contain a gene or genes that function in the biosynthesis of B6.

### Example 14

### Assay for the Detection of B6 Vitamers

An assay for the detection of B6 vitamers produced in heterologous systems can be performed by using Sacchromyces uvarum as an indicator strain described in Example 1. An alternative assay is an HPLC assay. Quantitation of B6 vitamers in supernatants of cultures of microorganisms or extracts of organisms that have been genetically modified to increase the production of B6 vitamers can be accomplished by filtration (Centricon, 0.45 μm), followed by high pressure liquid chromatography (HPLC). Quantitation is accomplished by comparing samples to standard curves generated by running a known solution of PMP, PLP, PNP, PM, PL or PN. For the HPLC assay, filtered supernatants are diluted 1/5 with 0.45 μm filtered Buffer A (50 mM KPO₄, 5 mM EDTA, 2% acetonitrile, pH 7.0) and 10 μL of this solution is injected onto a 250×4.6 mM Aqua™ 5μ C18 column run on an Agilent™ 1100 series HPLC equipped with a G1321A fluorescence detector. The excitation wavelength is 330 nM and the emission wavelength is 380 nM. An isocratic flow is maintained on the C18 column for 15 minutes with buffer A at 1 mL/min. This is followed by a 2 minute and a 15 minute 2% and 95% acetonitrile (in water without salts) wash, respectively. An equilibration step consisting of a 2% ACN wash for 5 min followed by a 5 minute Buffer A wash is performed in preparation for the next sample injection. Typical retention times for PMP, PM, PLP, PL and PN are 2.7, 4.6, 5.3, 7.8 and 9.6 minutes, respectively. Chem Station™, the accompanying software package provided by Agilent, is utilized for instrument control, data acquisition and data evaluation and run on a HP Pentium™ 4 computer that supports Microsoft™ Windows NT 4.0 updated with a Microsoft™ Service Pack (SP6a).

### Example 15

### Genes Homologous to the Type a B6 Biosynthetic Pathway Expressed in host organisms for the production of B6

Preferred methods for the production of vitamin B6 feature the expression of a gene of the type A pathway, e.g., epd, pdxA, pdxJ, pdxF, pdxB, pdxH or dxs homologs, or a combination thereof, in host organisms (type A or type B organisms) such as E. coli, S. cerevisiae, Arabidopsis thaliana, etc. In order to identify genes or proteins that are homologous to the type A pathway, a procedure such as a protein or nucleotide BLAST™ search can be implemented as described in Example 12 (Altschul, Stephen F. (1997) Nucleic Acids Res. 25: 3389-3402). Function can further be confirmed as described in Example 13.

Equivalents

Those skilled in the art will recognize, or be able to ascertain using no more than routine experimentation, many equivalents to the specific embodiments of the invention described herein. Such equivalents are intended to be encompassed by the following claims.

1. A method for producing a B6 vitamer comprising culturing an organism with an increased YaaD and/or YaaE activity as compared to the parent organism.

2. The method of claim 1, wherein increased YaaD and/or YaaE activity is due to increased expression of a nucleic acid molecule encoding a YaaD polypeptide and/or a YaaE polypeptide as compared to an unmodified parent organism.

3. The method of claim 2, wherein increased yaaD and/or yaaE nucleic acid molecule expression is due to the deregulation or introduction of nucleic acid molecules encoding a YaaD polypeptide and/or a YaaE polypeptide into the organism.

4. The method of claim 3, wherein said yaaD nucleic acid molecule encodes a polypeptide comprising the amino acid sequence of SEQ ID NO:21 and/or wherein said yaaE nucleic acid encodes a polypeptide comprising the amino acid sequence SEQ ID NO:23.

5. The method of claim 3, wherein said yaaD nucleic acid encodes a polypeptide comprising an amino acid sequence which is at least 30% identical to the amino acid sequence of SEQ ID NO:21, said polypeptide having a YaaD activity.

6. The method of claim 3, wherein said yaaE nucleic acid encodes a polypeptide comprising an amino acid sequence which is at least 30% identical to the amino acid sequence of SEQ ID NO:23, said polypeptide having a YaaE activity.

7. The method of claim 3, wherein a nucleic acid molecule comprising the nucleotide sequence of SEQ ID NO:20 and/or SEQ ID NO:22 is introduced.

8. The method of claim 1, wherein the organism is selected from the group consisting of plants and microorganisms.

9. The method of claim 8, wherein the microorganism is selected from the group consisting of algae, fungi, yeast, and bacteria.

10. A method for producing a B6 vitamer comprising culturing an organism with an increased Epd, PdxA, PdxJ, PdxF, PdxB, PdxH, and/or a Dxs activity as compared to the parent organism.

11. The method of claim 10, wherein increased Epd, PdxA, PdxJ, PdxF, PdxB, PdxH, and/or a Dxs activity is due to increased expression of a nucleic acid molecule encoding an Epd, PdxA, PdxJ, PdxF, PdxB, PdxH, and/or Dxs polypeptide as compared to an unmodified parent organism.

12. The method of claim 11, wherein increased epd, pdxA, pdxJ, pdxF, pdxB, pdxH, and/or dxs nucleic acid molecule expression is due to the introduction of nucleic acid molecules encoding an Epd, PdxA, PdxJ, PdxF, PdxB, PdxH, and/or Dxs polypeptide into the organism.

13. The method of claim 12, wherein said pdxA nucleic acid encodes a polypeptide comprising the amino acid sequence of SEQ ID NO:25 and/or wherein said pdxJ nucleic acid encodes a polypeptide comprising the amino acid sequence SEQ ID NO:27.

14. The method of claim 12, w herein said pdxA nucleic acid encodes a polypeptide comprising an amino acid sequence which is at least 30% identical to the amino acid sequence of SEQ ID NO:25, said polypeptide having a PdxA activity.

15. The method of claim 12, wherein said pdxJ nucleic acid encodes a polypeptide comprising an amino acid sequence which is at least 30% identical to the amino acid sequence of SEQ ID NO:27, said polypeptide having a PdxJ activity.

16. The method of claim 12, wherein a nucleic acid molecule comprising the nucleotide sequence of SEQ ID NO:24 and/or SEQ ID NO:26 is introduced.

17. The method of claim 10, wherein the organism is selected from the group consisting of plants and microorganisms.

18. The method of claim 17, wherein the microorganism is selected from the group consisting of algae, fungi, yeast, and bacteria.

19. An organism that has been genetically modified to comprise a recombinant DNA molecule that results in the increase of the activity of one or more enzymes that catalyze(s) a step in the biosynthesis of a B6 vitamer, such that B6 vitamer production from said modified organism is increased compared to B6 production in an unmodified parent organism.

20. The organism of claim 19, wherein B6 vitamer production is at least ten-fold higher than from the unmodified parent organism.

21. The organism of claim 19, wherein said enzyme is one or more of YaaD or YaaE, or a homologue thereof, wherein said homologue has the ability to rescue an auxotroph in a test system.

22. The organism of claim 21, wherein said homologue is selected from the homologues listed in Table 9 or Table 10.

23. The organism of claim 19, wherein said organism is genetically modified to overexpress one or more genes that encodes an enzyme that catalyzes a step in the biosynthesis of a B6 vitamer.

24. The organism of claim 23, wherein said genes are derived from Bacillus.

25. The organism of claim 23, wherein said genes are derived from Bacillus subtilis.

26. The organism of claim 23, wherein at least one of said genes is a yaaD gene.

27. The organism of claim 23, wherein at least one of said genes is a yaaE gene.

28. The organism of claim 23, wherein at least two of said genes are yaaD and yaaE genes.

29. The organism of claim 23, wherein said organism is a Bacillus strain.

30. The organism of claim 23, wherein said organism is Bacillus subtilis.

31. The organism of claim 23, wherein said organism is Escherichia coli.

32. The organism of claim 19, wherein said genes are selected from the group consisting of E. coli epd, pdxA, pdxJ, pdxF, pdxB, pdxH or dxs.

33. The organism of any one of claims 19-30, wherein said organism is grown in a liquid culture and the total B6 vitamer concentration in the culture supernatant is at least 7.0 mg/liter.

34. A method of producing a B6 vitamer comprising culturing a microorganism that has been genetically modified to overexpress one or more genes that encodes an enzyme that catalyzes a step in the biosynthesis of a B6 vitamer, such that B6 vitamer production from said modified organism is increased compared to B6 production in an unmodified parent organism, under conditions such that the B6 vitamer is produced.

35. The method of claim 34, wherein said enzyme is one or more of YaaD or YaaE.

36. The method of claim 34, wherein at least one of said genes is a yaaD gene.

37. The method of claim 34, wherein at least one of said genes is a yaaE gene.

38. The method of claim 34, wherein said genes are contained on the yaaDE operon.

39. The method of claim 34, wherein the B6 vitamer is pyridoxine.

40. The method of claim 34, wherein the B6 vitamer is pyridoxal.

41. The method of claim 34, wherein the B6 vitamer is pyridoxamine.

42. The method of claim 34, wherein said genes are bacterial-derived.

43. The method of claim 34, wherein said genes are derived from Bacillus.

44. The method of claim 34, wherein said genes are derived from Bacillus subtilis.

45. The method of claim 34, wherein the microorganism is Gram positive.

46. The method of claim 34, wherein the microorganism is a microorganism belonging to a genus selected from the group consisting of Bacillus, Cornyebacterium, Lactobacillus, Lactococci and Streptomyces.

47. The method of claim 34, wherein the microorganism is of the genus Bacillus.

48. The method of claim 34, wherein the microorganism is Bacillus subtilis.

49. The method of claim 34, further comprising recovering the B6 vitamer.

50. A method of producing a B6 vitamer comprising culturing a microorganism that overexpresses at least one Bacillus B6 vitamer biosynthetic gene under conditions such that the B6 vitamer is produced.

51. The method of claim 50, wherein the microorganism overexpresses at least one Bacillus subtilis B6 vitamer biosynthetic enzyme.

52. The method of claim 50, wherein the B6 vitamer is pyridoxine.

53. The method of claim 50, wherein the B6 vitamer is pyridoxal.

54. The method of claim 50, wherein the B6 vitamer is pyridoxamine.

55. The method of claim 50, wherein the microorganism overexpresses at least two B6 vitamer biosynthetic enzymes.

56. The method of claim 50, wherein the microorganism is Gram positive.

57. The method of claim 50, wherein the microorganism is Gram negative.

58. The method of claim 50, wherein the microorganism is a microorganism belonging to a genus selected from the group consisting of Bacillus, Cornyebacterium, Lactobacillus, Lactococci and Streptomyces.

59. The method of claim 50, wherein the microorganism is of the genus Bacillus.

60. The method of claim 50, wherein the microorganism is Bacillus subtilis.

61. The method of claim 50, further comprising recovering the B6 vitamer.

62. A recombinant microorganism that overexpresses at least one Bacillus B6 vitamer biosynthetic gene.

63. A recombinant microorganism that overexpresses at least one Bacillus B6 vitamer biosynthetic enzyme.

64. The method of claim 63, wherein said enzyme is YaaD or YaaE.

65. The recombinant microorganism of claim 62 that overexpresses at least one Bacillus subtilis B6 vitamer biosynthetic gene.

66. The recombinant microorganism of claim 62, wherein the B6 vitamer biosynthetic gene is selected from the group consisting of yaaD and yaaE.

67. The recombinant microorganism of claim 62, that is Gram positive.

68. The recombinant microorganism of claim 62, belonging to a genus selected from the group consisting of Bacillus, Cornyebacterium, Lactobacillus, Lactococci and Streptomyces.

69. The recombinant microorganism of claim 62 belonging to the genus Bacillus.

70. The recombinant microorganism of claim 62 which is Bacillus subtilis.

71. A recombinant microorganism selected from the group consisting of PX14 and PX17.

72. A vector comprising a nucleic acid sequence that encodes at least one Bacillus B6 vitamer biosynthetic gene operably linked to regulatory sequences.

73. The vector of claim 72, comprising a nucleic acid sequence that encodes at least one Bacillus subtilis B6 vitamer biosynthetic gene.

74. The vector of claim 72, wherein the regulatory sequences comprise a constitutively active promoter.

75. The vector of claim 72, wherein the constitutively active promoter comprises P₁₅ (SEQ ID NO:9) or P₂₆ (SEQ ID NO: 10) sequences.

76. The vector of claim 72, wherein the regulatory sequences comprise at least one artificial ribosome binding site (RBS).

77. A vector selected from the group consisting of pDX14R and pDX17R.

78. A recombinant microorganism comprising the vector of claim 72.

79. An isolated nucleic acid molecule that encodes at least one Bacillus B6 vitamer biosynthetic gene.

80. The isolated nucleic acid molecule of claim 79 that encodes at least one Bacillus subtilis B6 vitamer biosynthetic gene.

81. An isolated Bacillus B6 vitamer biosynthetic enzyme polypeptide.

82. An isolated Bacillus subtilis B6 vitamer biosynthetic enzyme polypeptide.


SEQUENCE LISTING (27 sequences)

SEQ ID NO: 1 - DNA, 46 bp, Artificial Sequence
  Description of Artificial Sequence Synthetic Primer
ccctctagaggaggagaaaacatggctcaaacaggtactgaacgtg

SEQ ID NO: 2 - DNA, 45 bp, Artificial Sequence
  Description of Artificial Sequence Synthetic Primer
cccggatcctcaactgttttatacaagtgccttttgcttatattc

SEQ ID NO: 3 - DNA, 49 bp, Artificial Sequence
  Description of Artificial Sequence Synthetic Primer
ccctctagaggaggagacataatggaacgtacaacgaattttaacgcag

SEQ ID NO: 4 - DNA, 41 bp, Artificial Sequence
  Description of Artificial Sequence Synthetic Primer
cccggatcccggacggtttgcatagccagactttttactca

SEQ ID NO: 5 - DNA, 5322 bp, Artificial Sequence
  Description of Artificial Sequence plasmid - pDX1F
tgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggatccctctagaggaggagaaaacatggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttatcactagtgcggccgcctgcaggtcgaccatatgggagaggcggccgcgtcgaccaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccggtaccgggctcccaacgcgttggatgcatagcttgagtattctatagtgtcacctaaatagcttggcgtaatcatggtcatagctgtttcctgtgtgaaattgttatccgaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgtataaaacagttgaggatccgggatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 6 - DNA, 5297 bp, Artificial Sequence
  Description of Artificial Sequence plasmid - pDX11F
tgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggatccctctagaggaggagacataatggaacgtacaacgaattttaacgcaggtcctgcagcgctgccactggaagttctgcaaaaagcacagaaagaatttattgattttaacgaatccggcatgtctgttatggagctttcccaccgcagcaaagagtatgaagcggtgcaccaaaaagcgaaaagcctcttaatcgaactgatgggcattccggaagattacgatatcttgtttcttcaaggcggggcaagccttcaattctcaatgcttccgatgaactttttaacacctgaaaaaaccgcacattttgtgatgaccggcgcttggtctgaaaaagcactggcagaaacgaaactgttcgggaacacgtctatcaccgctacaagtgaaacagacaattacagttatattccagaggttgaccttacggatgtaaaagacggcgcatatttacatatcacatccaacaatacaattttcggcactcagtggcaggagtttccgaattctccaattccgctcgtagccgacatgtccagcgatattttaagcagaaaaatcgatgtgtccaaatttgatgtgatctacggaggcatcactagtgcggccgcctgcaggtcgaccatatgggagaggcggccgcgtcgaccaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccggtaccgggctcccaacgcgttggatgcatagcttgagtattctatagtgtcacctaaatagcttggcgtaatcatggtcatagctgtttcctgtgtgaaattgttatccgtagtcatcatgaaaaaaagctggctgcaaaatgaaaatgcgaacgtcccaaaaatcttgaaatattccacgcatgtcaaagcggattcactctacaacactccgccgacatttgcgatttatatgctgagcctcgttctggaatggctcaaggaaaacggcggtgtggaagctgttgaacagcgcaatgaacaaaaagcgcaggttctctacagctgtattgatgaaagcaacggcttctataaaggacatgccagaaaagacagccgctcacgcatgaatgtcacattcacgcttcgggatgacgaattaacgaaaacattcgttcagaaagcaaaagatgcgaagatgatcggccttggcggacaccgttcggtgggaggctgccgcgcttctatttataacgcggtctctctcgaagactgtgaaaaattagctgcgttcatgaagaaattccagcaggaaaatgagtaaaaagtctggctatgcattccgtccgggatccgggatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 7 - DNA, 6731 bp, Artificial Sequence
  Description of Artificial Sequence plasmid - pDX14R
ttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatcctcaactgttttatacaagtgccttttgcttatattcctcaaccatttcaacaaacagctgcgtcactcggtgatcttctgtcagctccggatggaatgagcagccaaggaattgcccctgtttcgcggctacaatacgaccattatgctccgatagaacttcaacattttcaccagcttctaaaatatgcggagcacggatgaataccccagtaaaaggctcgtccaagcctttaattgttaaatcagcttcaaagctgtcaacctgccggccaaatgaattacgttctacaaccacattcagaagacctaaatgaggattatctgaaccggcaatttcttttgctaatataattaatccggcacatgttccaaacatcggtttgccctgagcagcgaattcacgaagcggctccatgaattgatacgtatcgatcaaacggcgcatcgtcgtgctctcaccgcccggcaaaatcaacccgtcaacttcgttcagctgctccggacgttttacgacaagaccagccgcgccgcatgcttcaatcgcatggatgtgctctctaactgctccttgaagtcctagtacacctattgttaacatgtcagcagcgctcctatgttcttaccagccgcgttcttgcatacgctgttctggaagtaagtttgagatttcaatccctttcattgcagtaccaagctcttttgacaactcagcgattaatttgtaatcagtaaagtgagttgttgcttccacaattgctttcgcaaatttagcagggttgtctgatttaaaaataccagaaccaacaaatactccgtcagcaccaagctgcatcatgagagcagcatcagctggagttgctacgccgccagcggcaaagttaacgacaggaagcttgccgtcttttttaatttgaagaagaagctcgtaaggagcacctaggtttttcgcttctgtcattagctcatcctcactcatcgcaactactttgcgcacttgagcgttaactttacgcatatggcgaacagcctcaacaatattacctgttccaggctcaccttttgtgcgaagcatagaagcaccttccgcaatacggcgtgttgcttcaccaagatcacggcagccacagacaaaaggaactgtgtattcatttttatttaaatgaaattcttcgtcagccggcgtcagaacttcactttcatcaatatagtcaacacccatagcttcaagcacacgcgcttcaacaatatgtccgatacgcgcttttgccattaccgggatagatactgcattcattacttcttccacgattgtagggtcagccatacgggcaactcctccagccgcgcgaatatctgctggcacacgttctagcgccattacagcgacagctccagcttcttcagcgattttcgcttgttccgcattgatgacgtccatgatgacgccgcctttttgcatttctgccattccgcgttttacacgttcagtacctgtttgagccatgttttctcctcctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacctgcaggcatgcaagcttcgcgaagcggccgccgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 8 - DNA, 10181 bp, Artificial Sequence
  Description of Artificial Sequence plasmid - pDX17R
gaattttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatcctcaactgttttatacaagtgccttttgcttatattcctcaaccatttcaacaaacagctgcgtcactcggtgatcttctgtcagctccggatggaatgagcagccaaggaattgcccctgtttcgcggctacaatacgaccattatgctccgatagaacttcaacattttcaccagcttctaaaatatgcggagcacggatgaataccccagtaaaaggctcgtccaagcctttaattgttaaatcagcttcaaagctgtcaacctgccggccaaatgaattacgttctacaaccacattcagaagacctaaatgaggattatctgaaccggcaatttcttttgctaatataattaatccggcacatgttccaaacatcggtttgccctgagcagcgaattcacgaagcggctccatgaattgatacgtatcgatcaaacggcgcatcgtcgtgctctcaccgcccggcaaaatcaacccgtcaacttcgttcagctgctccggacgttttacgacaagaccagccgcgccgcatgcttcaatcgcatggatgtgctctctaactgctccttgaagtcctagtacacctattgttaacatgtcagcagcgctcctatgttcttaccagccgcgttcttgcatacgctgttctggaagtaagtttgagatttcaatccctttcattgcagtaccaagctcttttgacaactcagcgattaatttgtaatcagtaaagtgagttgttgcttccacaattgctttcgcaaatttagcagggttgtctgatttaaaaataccagaaccaacaaatactccgtcagcaccaagctgcatcatgagagcagcatcagctggagttgctacgccgccagcggcaaagttaacgacaggaagcttgccgtcttttttaatttgaagaagaagctcgtaaggagcacctaggtttttcgcttctgtcattagctcatcctcactcatcgcaactactttgcgcacttgagcgttaactttacgcatatggcgaacagcctcaacaatattacctgttccaggctcaccttttgtgcgaagcatagaagcaccttccgcaatacggcgtgttgcttcaccaagatcacggcagccacagacaaaaggaactgtgtattcatttttatttaaatgaaattcttcgtcagccggcgtcagaacttcactttcatcaatatagtcaacacccatagcttcaagcacacgcgcttcaacaatatgtccgatacgcgcttttgccattaccgggatagatactgcattcattacttcttccacgattgtagggtcagccatacgggcaactcctccagccgcgcgaatatctgctggcacacgttctagcgccattacagcgacagctccagcttcttcagcgattttcgcttgttccgcattgatgacgtccatgatgacgccgcctttttgcatttctgccattccgcgttttacacgttcagtacctgtttgagccatgttttctcctcctctagaacaggcggggttgcccccgcctgtaattaaattattacacaccctgtagggaaagtcaatacctttttgtaaaatttttacacagcgtggatctcttctagggacacctctttgtacccctcaagggagaaatattggcggtactgagcacagttttggttggtggacagtgaaccatagctgtcgtcaatagcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacgatgatctgccgttttcttctgcaagccaaaaaaccttccgttacaacgagaaggattcttcactttctaaagttcggcgagtttcatccctctgtcccagtccttttttggatcaaggcagactgctgcaatgtctatctattttaataataggtgcagttcgcaggcgatactgcccaatggaagtataccaaaatcaacgggcttgtaccaacacattagcccaattcgatatcggcagaatagatttttttaatgccttcgttcgtttctaaaagcagaacgccttcatcatctatacctaacgccttaccgtaaaaggttccgtttaacgttctggctctcatattagtgccaataccgagcgcatagctttcccataaaagcttaatcggcgtaaatccgtgcgtcatataatcccggtaccgtttctcaaagcatagtaaaatatgctggatgacgccggcccgatcaattttttccccagcagcttggctgaggcttgtcgcgatgtccttcaattcatctggaaaatcattaggctgctggttaacgttaatgccgatcccaatgatcactgaacgtacgcggtcttcttcagcctgcatttccgttaggataccgactgttttttttccgttaatcaaaatatcatttggccatttaatatccgtttggatgcctgctgcctcttctattccctgcacaacagctactgcagcaagcagagtcagctgcggtgttttttggagcggaatgtcaggccgcaaaatcaggctcatccaaacaccgtttccttcttgagaatgccataccctagacattcggccccttccggctgtttgtttgtcagccaccacaagggtgccttccggtgcgttattattcgcgagctcatgagccgttttttgcgtgcttgaaagaacgtcatggtaaataagatgctggcccatcacttccgtttttaatccaaaacgaatttcgctttcactgagttttccgggttttttgatgagccgatatccttttcttctaacggcttctacttcataaccctctttccgaagctcttcaatatgcttccacacagcagttcttgaacagccgagagcatcactgattttttggccggaaataaattcattgccggcctgagaaaataattcaataaggtcttttcttaatgttgaccgcatgtcttcagccactcctctatgtgtttcttttgattggagagcttccctgtcacaacagcctgctcgatccactgtaattcttctgacacccattttccggccggccggtttcgaagcgcaagcaagtccttacccgtgatatcaagatccttaaggcttttgatcggcaggttttgataagcgtactgaatgtccttcagtttcttttcatccagtttttcgttttgccgaagctgcgatattttggccgctgagagcagtgcttttttcccagctctgtacattgtcattgcgtcaaggctctggccaaacgtatcggcaatgtgaatggcttccttgatcacttttcccgggagcttccaggctttcaggaaaaggggcgcgtctttcaaaactatgccaaggttaattaaaagagcagcccaaagctcctcacgggatgttaaagagaagaatggaaactcactcgttgaaatcaggttttctcgtttatgataaaaaccaggaagctcttcatacaatctcgtttgaatgagtgtttgaagcgcctggcgagaagctcttccctgcagcaatttctcaaactctatagtttttcgttcgactgaaacatgggagaggagtgatttttctttcgcaatggcttcttctgtttccggtgaaagcgtaaagccaagctggctcataaagcgtacggctctcagcatacgaagcgcatcctcttgaaatctatcctcaggctttccaacggttcgaatcactttctgatcaatatctttcttgccgccaaaataatcaagcaccttcccgtccgctgtcatggccatcgcattgatcgttaaatctctgcgttttagatcctcttctaatgatgagataaattgcacttctgacggtcttctgaaatcaacataatcagattcagtccggaatgtcgtgacttcataggtttcatcctcccagagcacaataatggtcccgtgctctttgcctacatcaacagtccgctgaaacagccgttctacttgatcaggtgccgcatctgtcgcgatatcgacatctccgatcgttcgtttcatatagctgtcacgaactgcgcccccgacaaaataagcctgatggcccgcttcgattaagatgcggagcacgggaagtgctttgataaaaactttttccatgtgatcactccggttctgctaaatcggcataaatctgttcatactggctgacaatttttttagaagaaaattcattttcaagcatctctattgccgcctttgtaaaacgattgcttagctgttcatcttctaaaatgctcatcgcgcgggctgttgcggccgtaacatcaccgacatccaccaaaaatccgctcacattgttttttataacctcagggataccgccaatgtttgttccaatacaaggcactccgcaagccatcgcttcaagcaggacaaggccaaagctttctttttcagatagcagcagcttcaaatcgctaatagaataaagatcttcaacacggtcttgatttccaagcattaagacttggtcttccaagccatattttctgataagctcgcaggctgtcgatttctccggaccgtctccgactaaaagcagcttcgctttcgttttgccagcgatattgcggaacacacggatgacatcctgcacgcgtgcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaac

SEQ ID NO: 9 - DNA, 194 bp, Artificial Sequence
  Description of Artificial Sequence Artificial Promoter
gctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgt

SEQ ID NO: 10 - DNA, 163 bp, Artificial Sequence
  Description of Artificial Sequence Artificial Promoter
gcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgc

SEQ ID NO: 11 - DNA, 127 bp, Artificial Sequence
  Description of Artificial Sequence Artificial Promoter
gaggaatcatagaattttgtcaaaataattttattgacaacgtcttattaacgttgatataatttaaattttatttgacaaaaatgggctcgtgttgtacaataaatgtagtgaggtggatgcaatg

SEQ ID NO: 12 - DNA, 42 bp, Bacillus subtilis
gaaatcatataactataccttgattagggggaccaagaaatg

SEQ ID NO: 13 - DNA, 42 bp, Bacillus subtilis
caagaacgcggctggtaagaacataggagcgctgctgacatg

SEQ ID NO: 14 - DNA, 16 bp, Artificial Sequence
  Description of Artificial Sequence Artificial ribosome binding site
tctagaaaggaggtga

SEQ ID NO: 15 - DNA, 22 bp, Artificial Sequence
  Description of Artificial Sequence Artificial ribosome binding site
tctagaaggaggagaaaacatg

SEQ ID NO: 16 - DNA, 21 bp, Artificial Sequence
  Description of Artificial Sequence Artificial ribosome binding site
tctagaggaggagaaaacatg

SEQ ID NO: 17 - DNA, 27 bp, Artificial Sequence
  Description of Artificial Sequence Artificial ribosome binding site
taagaacaaaggaggagagctgacatg

SEQ ID NO: 18 - DNA, 27 bp, Artificial Sequence
  Description of Artificial Sequence Artificial ribosome binding site
taagaacagaggaggagagctgacatg

SEQ ID NO: 19 - DNA, 27 bp, Artificial Sequence
  Description of Artificial Sequence Artificial ribosome binding site
taagaagaaaggaggtgagctgacatg

SEQ ID NO: 20 - DNA, 1282 bp, Bacillus subtilis
ttaatcaattgaaagagctctgatggatgttagggctctttcgcgtaatatactgaaaaaacccaatttttaataaaaaatgattttcaaagcgctaaaaaccgaataaaatagtatatatgcatatgaattattggatttctaggatacaataaggattagaaatcatataactataccttgattagggggaccaagaaatggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggtaagaacataggagcgctgctgacatgttaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgta

SEQ ID NO: 21 - protein, 293 aa, Bacillus subtilis
AQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 22 - DNA, 988 bp, Bacillus subtilis
ctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggtaagaacataggagcgctgctgacatgttaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgtataaaacagttgaaagctgtggaaacttatagtacattataagcacaaataaagatcgaaaagcgttgataggaactagtagggagcctctctttctaagagagccgatggttggtgcgaatcggtgaaagatgctgtctgaatccatccttgagcgaaatgctgaagcgagtaggcatttacgggataaccgttatgttt

SEQ ID NO: 23 - protein, 196 aa, Bacillus subtilis
MLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 24 - DNA, 990 bp, Escherichia coli
atggttaaaacccaacgtgttgtgatcactcccggcgagcccgccgggattggcccggacttagttgtccagcttgcacagcgtgagtggccggtcgaactggttgtttgtgccgatgccactctccttaccaaccgggcagcgatgctcggtttgccgctcaccctccgcccttattcccccaactcccctgcacaaccgcaaactgcgggcacattaacgctacttcctgtcgcgctacgtgcacctgtcactgcggggcagttagcggttgaaaatgggcattatgtggtggaaacgctggcgcgagcgtgcgatggttgtctgaacggcgaatttgccgcgctgatcacaggtccggtgcataaaggcgttattaacgacgctggcattccttttaccggtcataccgagtttttcgaagagcgttcgcaggcgaaaaaggtggtgatgatgctggcgaccgaagaacttcgcgtggcgctggcaacgacgcatttaccgctgcgcgatatcgcagacgctatcacccctgcacttttgcacgaagtgattgctattttgcatcacgatttgcggaccaaatttggtattgccgaaccgcgcattctggtctgcgggctgaatccgcacgcgggcgaaggcggtcatatgggtacggaagagatagacaccattattccggtgctcaatgagctgcgggcgcaggggatgaaactcaacgggccgctgcctgccgataccctgtttcagccgaaatatcttgataacgccgacgccgtgctggcgatgtaccacgatcagggtcttcccgtgctaaaataccagggcttcgggcgcggtgtgaacattacgctgggcctgccctttattcgcacatcagtggaccacggcaccgcgcttgaactggcgggacgtggcaaagccgatgtcggcagttttattacggcgcttaatctcgccatcaaaatgattgttaacacccaatga

SEQ ID NO: 25 - protein, 329 aa, Escherichia coli
MVKTQRVVITPGEPAGIGPDLVVQLAQREWPVELVVCADATLLTNRAAMLGLPLTLRPYSPNSPAQPQTAGTLTLLPVALRAPVTAGQLAVENGHYVVETLARACDGCLNGEFAALITGPVHKGVINDAGIPFTGHTEFFEERSQAKKVVMMLATEELRVALATTHLPLRDIADAITPALLHEVIAILHHDLRTKFGIAEPRILVCGLNPHAGEGGHMGTEEIDTIIPVLNELRAQGMKLNGPLPADTLFQPKYLDNADAVLAMYHDQGLPVLKYQGFGRGVNITLGLPFIRTSVDHGTALELAGRGKADVGSFITALNLAIKMIVNTQ

SEQ ID NO: 26 - DNA, 732 bp, Escherichia coli
atggctgaattactgttaggcgtcaacattgaccatatcgctacgctgcgcaacgcgcgcggtaccgcttacccggatccggtgcaggccgcgtttattgccgagcaggcgggagcggacggcattaccgtgcatttacgtgaagatcgccgtcacattactgaccgcgacgtgcgcatcctgcgtcagacgctggatacccgcatgaatctggagatggcggtgaccgaagagatgctggcgatcgccgttgagacgaagccacatttttgctgcctggtaccggaaaagcgtcaggaagtaacaaccgaaggcggcctggatgtcgcagggcagcgtgacaaaatgcgcgatgcctgcaaacgtctggcagatgccgggattcaggtttctctgtttattgacgccgatgaagagcagatcaaagctgcggcagaggttggcgcaccgtttatcgagatccacaccggttgctatgctgatgccaaaactgacgccgaacaggcgcaagagctggcgcgtatcgccaaagccgcgacctttgccgcaagcctcggtctgaaagttaacgccggacacggtctgacctatcacaacgtgaaagccattgccgccatccctgagatgcatgaactgaatatcggtcatgccattattggtcgtgcagtgatgaccggactgaaagatgcggtggcagaaatgaagcgtctgatgctggaagcgcgtggctaa

SEQ ID NO: 27 - protein, 243 aa, Escherichia coli
MAELLLGVNIDHIATLRNARGTAYPDPVQAAFIAEQAGADGITVHLREDRRHITDRDVRILRQTLDTRMNLEMAVTEEMLAIAVETKPHFCCLVPEKRQEVTTEGGLDVAGQRDKMRDACKRLADAGIQVSLFIDADEEQIKAAAEVGAPFIEIHTGCYADAKTDAEQAQELARIAKAATFAASLGLKVNAGHGLTYHNVKAIAAIPEMHELNIGHAIIGRAVMTGLKDAVAEMKRLMLEARG